(19) United States
(12) Patent Application Publication
(10) Pub. No.: US 2021/0322458 A1
(43) Pub. Date: Oct. 21, 2021

(54) METHODS AND COMPOSITIONS FOR TREATMENT OF BURNS, JOINT PAIN, AND FUNGAL INFECTIONS

(71) Applicants: Gregg Tobin, Alpharetta, GA (US); Greg Glaze, Alpharetta, GA (US)

(72) Inventors: Gregg Tobin, Alpharetta, GA (US); Greg Glaze, Alpharetta, GA (US)

(21) Appl. No.: 17/235,165

(22) Filed: Apr. 20, 2021

Related U.S. Application Data
(60) Provisional application No. 63/134,478, filed on Jan. 6, 2021.
(60) Provisional application No. 63/114,085, filed on Nov. 16, 2020.
(60) Provisional application No. 63/012,474, filed on Apr. 20, 2020.

(51) Int. Cl. A61K 31/728; A61K 36/886; A61K 33/38; A61K 31/137; A61K 31/27; A61K 45/06

(52) U.S. Cl. CPC A61K 31/728; A61K 36/886; A61K 45/06; A61K 31/137; A61K 31/27; A61K 33/38

(57) ABSTRACT

In one aspect, the disclosure relates to pharmaceutical compositions for treatment of burns, e.g., sunburns, methods of making same, and methods of treating burns using same. The disclosure, in another aspect, relates to pharmaceutical compositions for treatment of joint pain, methods of making same, and methods of treating joint pain using same. This abstract is intended as a scanning tool for purposes of searching in the particular art and is not intended to be limiting of the present disclosure.

### CROSS REFERENCE TO RELATED APPLICATIONS

This application claims the benefit of U.S. Provisional Application No. 63/012,474 filed on Apr. 20, 2020; U.S. Provisional Application No. 63/114,085 filed on Nov. 16, 2020; and U.S. Provisional Application No. 63/134,478 filed on Jan. 6, 2021, each of which is incorporated herein by reference in their entireties.

### BACKGROUND

A burn is a type of injury to skin caused by heat, electricity, chemicals, friction, lasers, ice or radiation. Burns that affect only the superficial skin are known as superficial or first-degree burns. When damage penetrates into some of the underlying layers, it is a partial-thickness or second-degree burn. In a full-thickness or third-degree burn, the injury extends to all layers of the skin. A fourth-degree burn involves injury to deeper tissues, such as muscle or bone.

Sunburn is an acute reaction by the skin to excessive exposure to sunlight. Ordinary sunburn results from overexposure of the skin to ultraviolet waves of about 3000 Angstroms. Sunburn symptoms appear in 1 to 24 hours and, except in severe reactions, pass their peak in 72 hours. Following exposure to sunlight, the epidermis thickens and the melanocytes produce melanin at an increased rate, providing some natural protection against further exposure. Skin changes range from a mild erythema with subsequent evanescent scaling, to pain, swelling, skin tenderness, and blisters from more prolonged exposure. Fever, chills, weakness, and shock may appear if a large portion of the body surface is affected. Secondary infection, particularly furunculosis, is the most common late complication. The skin may remain hypervulnerable to sunlight for one to several weeks when pronounced exfoliation has occurred.

While treatments have been developed to treat bruises and burns, they have varying degrees of effectiveness. In the case of bruises from facial injections and facial surgery, for example, the dark puffiness associated with the bruise can not only be painful, but also very embarrassing. There are anecdotal reports that Arnica taken before surgery can help reduce bruising but has little effect on the bruise once the bruise has occurred. For that reason, more effective treatments are needed to quickly minimize the effects of bruises and burns.

Joint pain can occur due to a number of medical conditions such as autoimmune diseases including ankylosing spondylitis, rheumatoid arthritis, psoriatic arthritis, lupus, and others, or can be an effect of nutritional deficiencies (e.g. rickets), injuries (e.g., broken bones and dislocations), cancers (e.g., leukemia, bone cancer), a dysfunctional nervous system (e.g., complex regional pain system), other inflammatory conditions, and combinations thereof. Joint pain, whether temporary or long term, can affect an individual's quality of life, motor skills, and mobility. Oral pain medications can have adverse effects including addiction and/or abuse, as with opioids, liver damage when used in combination with alcohol or other medications, as with acetaminophen, may interfere with or exacerbate the effects other medications a patient is taking (e.g. aspirin with warfarin), or may be contraindicated (e.g., ibuprofen in patients with hypertension). In addition, some pain medications may not provide enough relief to patients. For these reasons, more effective and/or supplementary treatments for joint pain are needed to improve patient quality of life.

Despite advances in burn research, there is still a scarcity of compositions for effective treatment of burns, particularly sunburn, that can accelerate healing, reduce pain and discomfort, and provide a positive user experience. Furthermore, despite advances in pain control research, there is still a scarcity of compositions, especially topical compositions, that are effective for the treatment of joint pain stemming from a variety of medical conditions and injuries. These needs and other needs are satisfied by the present disclosure.

### SUMMARY

In accordance with the purpose(s) of the present disclosure, as embodied and broadly described herein, the disclosure, in one aspect, relates to pharmaceutical compositions for treatment of burns, e.g., sunburns, methods of making same, and methods of treating burns using same. The disclosure, in another aspect, relates to pharmaceutical compositions for treatment of joint pain, methods of making same, and methods of treating joint pain using same.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described aspects are usable in all aspects of the disclosure taught herein. Furthermore, the individual features of the dependent claims, as well as all optional and preferred features and modifications of the described aspects are combinable and interchangeable with one another.

Additional advantages of the disclosure will be set forth in part in the description which follows, and in part will be obvious from the description, or can be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### DETAILED DESCRIPTION

Many modifications and other aspects disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific aspects disclosed and that modifications and other aspects are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual aspects described and illustrated herein has discrete components and features which may be readily separated from or combined with the features of any of the other several aspects without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present disclosure is not entitled to antedate such publication by virtue of prior disclosure. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a therapeutic agent,” “an excipient,” or “an anti-inflammatory agent,” including, but not limited to, two or more such therapeutic agents, excipients, or anti-inflammatory agents, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, “administering” can refer to an administration that is topical, transcutaneous, and/or transdermal. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, “therapeutic agent” can refer to any substance, compound, molecule, and the like, which can be biologically active or otherwise can induce a pharmacologic, biologic and/or physiologic effect on a subject to which it is administered to by local action. A therapeutic agent can be a primary active agent, or in other words, the component(s) of a composition to which the whole or part of the effect of the composition is attributed. A therapeutic agent can be a secondary therapeutic agent, or in other words, the component(s) of a composition to which an additional part and/or other effect of the composition is attributed. The term therefore encompasses those compounds or chemicals traditionally regarded as drugs. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, the term “subject” can be a mammal such as a human. The term does not denote a particular age or sex. Thus, adult and juvenile subjects, whether male or female, are intended to be covered. In one aspect, the subject is a mammal. A patient refers to a subject afflicted with a disease or disorder.

As used herein, the terms “treat,” “treating,” and “treatment” can refer generally to obtaining a desired pharmacological and/or physiological effect. The degree of treatment using the compositions described herein can be compared to a control (e.g., a subject that has not been administered a composition described herein). The effect can be, but does not necessarily have to be, prophylactic in terms of preventing or partially preventing a disease, symptom or condition thereof, such as a burn, e.g., a sunburn. The effect can be therapeutic in terms of a partial or complete cure of a disease, condition, symptom or adverse effect attributed to the disease, disorder, or condition. The term “treatment” as used herein can include any treatment of a burn, e.g., a sunburn, in a subject, particularly a human, and can include any one or more of the following: (a) preventing the disease or condition from occurring in a subject which may be predisposed to the disease but has not yet been diagnosed as having it; (b) inhibiting the disease or condition, i.e., arresting its development; and (c) relieving the disease or condition, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, the term “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the disclosure (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound and/or pharmaceutical composition, for example, can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

The term “pharmaceutically acceptable” describes a material that is not biologically or otherwise undesirable, i.e., without causing an unacceptable level of undesirable biological effects or interacting in a deleterious manner.

The term “pharmaceutically acceptable salts”, as used herein, means salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

It is understood, that unless otherwise specified, temperatures referred to herein are based on atmospheric pressure (i.e. one atmosphere).

Described herein are pharmaceutical compositions that have therapeutic or clinical utility. Also described herein are methods of administering the pharmaceutical compositions to a subject in need thereof. In an aspect, the subject can have a burn, e.g., a sunburn. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Therapeutic Agents

The therapeutic agents disclosed herein are components that are useful in the disclosed pharmaceutical compositions in obtaining the desired clinical and non-clinical benefits, e.g., accelerate healing, reduce pain, reduce discomfort, and sooth the skin or dermis. In some instances, a particular disclosed therapeutic agent may be considered an “active ingredient” by a drug regulatory agency such as the U.S. Food and Drug Administration. In other instances, a particular disclosed therapeutic agent may be considered an “inactive ingredient” by a drug regulatory agency such as the U.S. Food and Drug Administration, but is nevertheless referred to as a therapeutic agent for the purposes of the present disclosure because it has been determined to be a useful component in the disclosed pharmaceutical compositions.

In various aspects, the therapeutic agent useful in the disclosed pharmaceutical compositions can comprise a first therapeutic agent, a second therapeutic agent, a fourth therapeutic agent, and a fifth therapeutic agent, or a pharmaceutically acceptable salt thereof each therapeutic agent. In a still further aspect, the first therapeutic agent comprises a dermal restorative agent, the second therapeutic agent comprises a pain modulator agent, the third therapeutic agent comprises soothing agent, the fourth therapeutic agent comprises an anti-inflammatory agent, and the fifth therapeutic agent comprises a dermal health agent.

In various aspects, the dermal restorative agent comprises at least one hyaluronic acid composition (“HA”). HA is a linear-chain heteropolysaccharide consisting of alternating residues of D-glucuronic acid and N-acetyl-D-glucosamine, with a weight-average molecular weight of from about 400 and to about 3,000,000 Da, depending on the source of extraction and the preparation method used. HA can be obtained, for example, by extraction from rooster combs (EP 138572 B1), by fermentation (from Streptococcus), or by biosynthesis (from Bacillus). Physiologically, HA is believed to have multiple positive functions in a body, ranging from mechanical support for the cells of many tissues such as skin, tendons, muscles and cartilage to tissue hydration and joint lubrication. It is also known that HA, through its CD44 membrane receptor, is able to modulate many different cell physiology and biology processes, such as cell proliferation, migration and differentiation and angiogenesis. Without wishing to be bound by a particular theory, it is believed to have a role in the wound-healing process, at least in part, due its ability to induce fibroblast migration; moderate the inflammatory phase through the activity of free radical scavengers; and activate negative feedback mediated by interaction with specific receptors (Trabucchi et al., Int J Tissue React, 2002, 24, 65-71). Further, without wishing to be bound by a particular theory, it is believed to have a wound remodeling effect because it regulates fibrosis by modulating collagen secretion. Without wishing to be bound by a particular theory, it is believed to that the indirect effect of hyaluronic acid on pain is due, in part, to protection and attenuation of mechanical and chemical stresses transmitted to the nociceptive nerve endings of the inflamed intra-articular area (Gomis et al., Arthritis Rheumatism, 2004, 50, 314-326).

In various aspects, the HA used in the disclosed pharmaceutical compositions can be by biosynthesis or fermentation, and has a weight-average molecular weight of between about 0.1 and about 1000 kDa, between about 1 and about 500 kDa, between about 130 and about 230 kDa, between about 145 and about 210 kDa, and most preferably between about 160 and about 200 kDa. It can be purchased from numerous companies (e.g., Lifecore Biomedical; QP Corp.; Seikagaku; Shiseido; Fidia farmaceutici) which are able to provide HA with the desired MW specifications.

In a further aspect, the hyaluronic acid is used in the present pharmaceutical compositions at a concentration, based on the total weight of the pharmaceutical composition, ranging from about 0.1 to about 10 wt %; about 0.1 to about 7 wt %; about 0.1 to about 5 wt %; about 0.1 to about 4 wt %; about 0.1 to about 3 wt %; about 0.1 to about 2 wt %; about 0.1 to about 1 wt %; about 0.2 to about 10 wt %; about 0.2 to about 10 wt %; about 0.2 to about 7 wt %; about 0.2 to about 5 wt %; about 0.2 to about 4 wt %; about 0.2 to about 3 wt %; about 0.2 to about 2 wt %; about 0.2 to about 1 wt %; about 0.3 to about 10 wt %; about 0.3 to about 10 wt %; about 0.3 to about 7 wt %; about 0.3 to about 5 wt %; about 0.3 to about 4 wt %; about 0.3 to about 3 wt %; about 0.3 to about 2 wt %; about 0.3 to about 1 wt %; about 0.4 to about 10 wt %; about 0.4 to about 10 wt %; about 0.4 to about 7 wt %; about 0.4 to about 5 wt %; about 0.4 to about 4 wt %; about 0.4 to about 3 wt %; about 0.4 to about 2 wt %; about 0.4 to about 1 wt %; about 0.5 to about 10 wt %; about 0.5 to about 10 wt %; about 0.5 to about 7 wt %; about 0.5 to about 5 wt %; about 0.5 to about 4 wt %; about 0.5 to about 3 wt %; about 0.5 to about 2 wt %; about 0.5 to about 1 wt %; or a sub-range within the foregoing ranges; or any combination of individual values within the foregoing ranges.

In various aspects, the pain modulator agent comprises at least one anesthetic agent, e.g., a local anesthetic agent. Exemplary, but non-limiting, classes of anesthetic agents useful in the disclosed pharmaceutical compositions include esters (e.g., butacaine, chloroprocaine, cocaine, cyclomethycaine, hexylcaine, procaine, proparacaine, propoxycaine, tetracaine, benzocaine), amide (e.g., bupivacaine, dibucaine, etidocaine, lidocaine, mepivacaine, ropivacaine, prilocaine), dyclonine, pramoxine and the pharmaceutically acceptable salts of the above compounds. In a further aspect, the pain modulator agent comprises at least one anesthetic agent selected from lidocaine, prilocaine, scandicaine, etidocaine, bupivacaine, ropivacaine, levobupivacaine, articaine, mepivacaine, and combinations thereof. Further useful local anesthetics include lidocaine hydrochloride, benzocaine and the like. Other local anesthetics are described in Textbook of Organic Medicinal and Pharmaceutical Chemistry. 3rd edition, Wilson et al., Lippincott Pub., 1956, pp 421-435, which is incorporated by reference.

Local anesthetics generally consist of a lipophilic group (frequently an aromatic ring) connected by an intermediate chain (commonly including an ester or amide) to an ionizable group (usually a tertiary amine). Optimal activity requires a delicate balance between the lipophilic and hydrophilic strengths of these groups. Since ester links (as in procaine) are more prone to hydrolysis than amide links, esters usually have a shorter duration of action. (Miller & Hondeghem, (1995), “Local Anesthetics” in Basic &Clinical Pharmacology, 6th Edition, Ed. by Katzung).

In a particular aspect, the pain modulator agent comprises lidocaine, or a pharmaceutically acceptable salt thereof. Lidocaine has the chemical name 2-(diethylamino)-N-(2,6-dimethylphenyl)-acetamide and is freely lipid soluble. It is insoluble in water and generally utilizes ethanol or the like to obtain a liquid solution. However, its salt form, lidocaine-HCl, is very soluble in water and alcohol. In another aspect, the pain modulator agent can be pramoxine. In one aspect, pramoxine may be especially useful for treating nociceptive pain.

In a further aspect, the pain modulator agent is used in the present pharmaceutical compositions at a concentration, based on the total weight of the pharmaceutical composition, ranging from about 1 to about 20 wt %; about 2 to about 20 wt %; about 5 to about 20 wt %; about 6 to about 20 wt %; about 7 to about 20 wt %; about 8 to about 20 wt %; about 9 to about 20 wt %; about 10 to about 20 wt %; about 11 to about 20 wt %; about 12 to about 20 wt %; about 13 to about 20 wt %; about 14 to about 20 wt %; about 15 to about 20 wt %; about 16 to about 20 wt %; about 17 to about 20 wt %; about 18 to about 20 wt %; about 1 to about 17 wt %; about 2 to about 17 wt %; about 5 to about 17 wt %; about 6 to about 17 wt %; about 7 to about 17 wt %; about 8 to about 17 wt %; about 9 to about 17 wt %; about 10 to about 17 wt %; about 11 to about 17 wt %; about 12 to about 17 wt %; about 13 to about 17 wt %; about 14 to about 17 wt %; about 15 to about 17 wt %; about 1 to about 15 wt %; about 2 to about 15 wt %; about 5 to about 15 wt %; about 6 to about 15 wt %; about 7 to about 15 wt %; about 8 to about 15 wt %; about 9 to about 15 wt %; about 10 to about 15 wt %; about 11 to about 15 wt %; about 12 to about 15 wt %; about 13 to about 15 wt %; about 14 to about 15 wt %; about 1 to about 12.5 wt %; about 2 to about 12.5 wt %; about 5 to about 12.5 wt %; about 6 to about 12.5 wt %; about 7 to about 12.5 wt %; about 8 to about 12.5 wt %; about 9 to about 12.5 wt %; about 10 to about 12.5 wt %; about 11 to about 12.5 wt %; about 12 to about 12.5 wt %; about 1 to about 10 wt %; about 2 to about 10 wt %; about 5 to about 10 wt %; about 6 to about 10 wt %; about 7 to about 10 wt %; about 8 to about 10 wt %; about 9 to about 10 wt %; or a sub-range within the foregoing ranges; or any combination of individual values within the foregoing ranges.

In various aspects, the soothing agent comprises at least one aloe, e.g., an aloe gel, aloe paste, aloe extract, aloe powder, or combinations thereof. Aloe vera gel is separated from the rind of the Aloe vera plant by filleting an aloe leaf, separating the inner gel from the outer leaf rind and separating it from the yellow sap contained within the rind. The inner gel is then homogenized and available for use or further processing either as a concentrate by removal of water or more extensive processing to create an extract. The Aloe vera gel contains a variety of chemical substances with a large molecular weight complex carbohydrate, identified and given the United States Adopted Name (USAN), acemannan. Fundamentally, acemannan the high molecular weight carbohydrate polymer of the gel can be separated either by alcohol precipitation, column purification or ultra-filtration. Aloe vera gel and its principle extract including acemannan process for preparation and its uses have been described in U.S. Pat. Nos. 4,735,935, 4,851,224, 4,957,907, 4,959,214, 4,917,890, 4,966,892, 5,106,616, 5,118,673, 5,308,838, 5,441,943, 5,703,060, 5,760,102 and 5,902,796. The entire contents of each patent are hereby incorporated by reference. Multiple properties have been attributed to this compound, but the most predominant has been its immunomodulation function. Included as part of the immunomodulation property is the ability to stimulate release of primary growth factors necessary for optimal and accelerated wound healing. In addition to its immune modulation activities it has also been shown to have anti-inflammatory properties and aid in the control of pain. The use of an aloe extract acemannan gel as a component of the present disclosure provides multiple attributes for accelerated healing, inflammation and pain control when used with one or more therapeutic agents as disclosed herein. Aloe vera gel or its extract, bulk acetylated mannans from Aloe vera (acemannan) can be used in present disclosure and any one of these products would be suitable for use. An anhydrous form of Aloe vera gel extract can also be used in the present disclosure. Without wishing to be bound by a particular theory, it is believed that aloe polysaccharides can enhance the skin's immune function and management of inflammatory changes from UV irradiation and to provide anti-aging benefits for the skin. In another aspect, aloe and aloe extracts can provide a cooling sensation.

In a further aspect, the soothing agent is used in the present pharmaceutical compositions at a concentration, based on the total weight of the pharmaceutical composition, ranging from about 1 to about 75 wt %; about 5 to about 75 wt %; about 10 to about 75 wt %; about 15 to about 75 wt %; about 20 to about 75 wt %; about 25 to about 75 wt %; about 30 to about 75 wt %; about 35 to about 75 wt %; about 45 to about 75 wt %; about 50 to about 75 wt %; about 55 to about 75 wt %; about 60 to about 75 wt %; about 70 to about 75 wt %; about 1 to about 70 wt %; about 5 to about 70 wt %; about 10 to about 70 wt %; about 15 to about 70 wt %; about 20 to about 70 wt %; about 25 to about 70 wt %; about 30 to about 70 wt %; about 35 to about 70 wt %; about 45 to about 70 wt %; about 50 to about 70 wt %; about 55 to about 70 wt %; about 60 to about 70 wt %; about 1 to about 65 wt %; about 5 to about 65 wt %; about 10 to about 65 wt %; about 15 to about 65 wt %; about 20 to about 65 wt %; about 25 to about 65 wt %; about 30 to about 65 wt %; about 35 to about 65 wt %; about 45 to about 65 wt %; about 50 to about 65 wt %; about 55 to about 65 wt %; about 60 to about 65 wt %; about 1 to about 60 wt %; about 5 to about 60 wt %; about 10 to about 60 wt %; about 15 to about 60 wt %; about 20 to about 60 wt %; about 25 to about 60 wt %; about 30 to about 60 wt %; about 35 to about 60 wt %; about 45 to about 60 wt %; about 50 to about 60 wt %; about 55 to about 60 wt %; about 1 to about 55 wt %; about 5 to about 55 wt %; about 10 to about 55 wt %; about 15 to about 55 wt %; about 20 to about 55 wt %; about 25 to about 55 wt %; about 30 to about 55 wt %; about 35 to about 55 wt %; about 45 to about 55 wt %; about 50 to about 55 wt %; about 1 to about 50 wt %; about 5 to about 50 wt %; about 10 to about 50 wt %; about 15 to about 50 wt %; about 20 to about 50 wt %; about 25 to about 50 wt %; about 30 to about 50 wt %; about 35 to about 50 wt %; about 45 to about 50 wt %; about 1 to about 45 wt %; about 5 to about 45 wt %; about 10 to about 45 wt %; about 15 to about 45 wt %; about 20 to about 45 wt %; about 25 to about 45 wt %; about 30 to about 45 wt %; about 35 to about 45 wt %; about 1 to about 40 wt %; about 5 to about 40 wt %; about 10 to about 40 wt %; about 15 to about 40 wt %; about 20 to about 40 wt %; about 25 to about 40 wt %; about 30 to about 40 wt %; about 35 to about 40 wt %; about 1 to about 35 wt %; about 5 to about 35 wt %; about 10 to about 35 wt %; about 15 to about 35 wt %; about 20 to about 35 wt %; about 25 to about 35 wt %; about 30 to about 35 wt %; about 1 to about 30 wt %; about 5 to about 30 wt %; about 10 to about 30 wt %; about 15 to about 30 wt %; about 20 to about 30 wt %; about 25 to about 30 wt %; about 1 to about 25 wt %; about 5 to about 25 wt %; about 10 to about 25 wt %; about 15 to about 25 wt %; about 20 to about 25 wt %; about 1 to about 20 wt %; about 5 to about 20 wt %; about 10 to about 20 wt %; about 15 to about 20 wt %; or a sub-range within the foregoing ranges; or any combination of individual values within the foregoing ranges.

In a further aspect, the soothing agent comprises an Aloe barbadensis leaf composition, e.g., an Aloe leaf composition obtained from Aloe barbadensis leaves as a gel, juice, extract. In a still further aspect, the concentration of anhydrous Aloe vera gel extract is present in an amount by weight of the topical composition of from 0.01-1.0 wt %, from 0.05-0.3 wt %, and from 0.075-0.2 wt %. In a yet further aspect, the Aloe leaf composition comprises a dried aloe vera extract present in an amount of dried aloe vera extract from about 0.5 wt % to 20 wt %; from about 0.15 wt % to 40 wt %; from about 0.5 wt % to 20 wt %; from about 0.75 wt % to 10 wt %; from about 1.0 wt % to 5.0 wt %; from about 1.25 wt % to 2.5 wt %; or from about 1.5 wt % to 2 wt %.

In various aspects, the anti-inflammatory agent comprises at least one non-steroidal anti-inflammatory drug (NSAID). In a further aspect, a NSAIDs suitable for preparation of the disclosed pharmaceutical compositions can comprise at least one compound from the following classes: salicylate derivatives (such as acetosalicylate [aspirin]), propionic acid derivatives (such as ibuprofen), aniline derivatives (such as acetaminophen), pyrazole derivatives (such as phenylbutazone), N-arylanthranilic acid (or fenamates) derivatives (such as meclofenamate), indole derivatives (such as indomethacin), acetic acid derivatives (such as diclofenac), oxicam derivatives (such as piroxicam), terpene derivatives (such as bisabolol), glyoxylic acid derivatives (such as allantoin), and miscellaneous others (such as celecoxib). In a still further aspect, the NSAIDs suitable for preparation of the disclosed pharmaceutical compositions include, but are not limited to: acetic acid derivatives (e.g., diclofenac, etodolac, ketorolac, and bromfenac), propionic acid derivatives (e.g., ibuprofen, fenoprofen, fluriboprofen, ketoprofen, naproxen, suprofen), fenamates (e.g., meclofenamate, mefenamic acid), oxicam (e.g., piroxicam, meloxicam), indole derivatives (e.g., indomethacin, sulindac), pyrazolone derivatives (e.g., phenylbutazone, oxyphenbutazone), bisabolol, allantoin, tolmetin, celecoxib, and the pharmaceutically acceptable salts of the above compounds.

In a further aspect, a disclosed NSAID inhibits at least one cyclooxygenase (COX) enzymes. In a still further aspect, the NSAID is a COX-2 specific inhibitor (i.e., a compound that inhibits COX-2 with an IC₅₀ that is at least 50-fold lower than the IC₅₀ for COX-1). Exemplary, but non-limiting, COX-2 inhibitors include celecoxib (CELEBREX™), valdecoxib (BEXTRA™), lumiracoxib (PREXIGE™), etoricoxib (ARCOXIA™) and/or rofecoxib (VIOXX™).

In a further aspect, the disclosed NSAID is a salicylate. Salicylates include by are not limited to acetylsalicylic acid or aspirin, sodium salicylate, choline and magnesium salicylates (TRILISATE™), and salsalate (DISALCID™). In an alternative aspect, the NSAID can be allantoin.

In a further aspect, the NSAID used in the disclosed pharmaceutical compositions can comprise an NSAID selected from the list consisting of aspirin, diflunisal, salsalate, ibuprofen, dexibuprofen, naproxen, fenoprofen, ketoprofen, dexketoprofen, flurbiprofen, oxaprozin, loxoprofen, indomethacin, tolmetin, sulindac, etodolac, ketorolac, diclofenac, nabumetone, piroxicam, meloxicam, tenoxicam, droxicam, lornoxicam, isoxicam, mefenamic acid, meclofenamic acid, flufenamic acid, tolfenamic acid, celecoxib, rofecoxib, valdecoxib, parecoxib, lumiracoxib, etoricoxib, firocoxib, paracetamol, nimesulide, licofelone, lysine clonixinate, and hyperforin, calcitriol, and combinations thereof. In a still further aspect, the disclosed NSAIDs is diclofenac, ketorolac, or a combination thereof.

In a further aspect, the NSAID is used in the present pharmaceutical compositions at a concentration, based on the total weight of the composition, ranging from about 0.1 to about 10 wt %; about 0.1 to about 7 wt %; about 0.1 to about 5 wt %; about 0.1 to about 4 wt %; about 0.1 to about 3 wt %; about 0.1 to about 2 wt %; about 0.1 to about 1 wt %; about 0.2 to about 10 wt %; about 0.2 to about 10 wt %; about 0.2 to about 7 wt %; about 0.2 to about 5 wt %; about 0.2 to about 4 wt %; about 0.2 to about 3 wt %; about 0.2 to about 2 wt %; about 0.2 to about 1 wt %; about 0.3 to about 10 wt %; about 0.3 to about 10 wt %; about 0.3 to about 7 wt %; about 0.3 to about 5 wt %; about 0.3 to about 4 wt %; about 0.3 to about 3 wt %; about 0.3 to about 2 wt %; about 0.3 to about 1 wt %; about 0.4 to about 10 wt %; about 0.4 to about 10 wt %; about 0.4 to about 7 wt %; about 0.4 to about 5 wt %; about 0.4 to about 4 wt %; about 0.4 to about 3 wt %; about 0.4 to about 2 wt %; about 0.4 to about 1 wt %; about 0.5 to about 10 wt %; about 0.5 to about 10 wt %; about 0.5 to about 7 wt %; about 0.5 to about 5 wt %; about 0.5 to about 4 wt %; about 0.5 to about 3 wt %; about 0.5 to about 2 wt %; about 0.5 to about 1 wt %; or a sub-range within the foregoing ranges; or any combination of individual values within the foregoing ranges.

In some aspects, the disclosed compositions include at least one anti-microbial or wound-healing agent. In another aspect, the anti-microbial agent can be or include silver or a silver salt. In one aspect, the silver or silver salt can be in the form of silver nitrate, silver sulfadiazine, colloidal silver, nanocrystalline silver, silver chloride, silver-calcium-sodium phosphate, silver salicylic acid, silver charcoal, silver zeolite, or silver nanoparticles. In some aspects, when silver is present as a silver salt in the Ag⁺ oxidation state, the silver is soluble in aqueous environments. Without wishing to be bound by theory, silver ions can act via several mechanisms including, but not limited to, forming pores in bacterial cell walls by reacting with peptidoglycan, inhibiting cellular respiration and/or other metabolic pathways, and disrupting DNA replication. In some aspects, silver nanoparticles can have a high surface area to volume ratio, which can result in increased activity with low in vivo toxicity. In any of these aspects, silver can additionally assist in the formation of reactive oxygen species (ROS), which can further damage bacterial cells. In another aspect, silver may be especially useful for the treatment of burns and/or for preventing infections in burns.

In one aspect, when the silver is or includes silver sulfadiazine, the disclosed compositions include from about 0.5% to about 1.5% (w/w) silver sulfadiazine, or about 1% (w/w) silver sulfadiazine, or a combination of any of the foregoing values, or a range encompassing any of the foregoing values. In another aspect, when the silver is or includes silver nitrate, the disclosed compositions include from about 0.01% to about 1% (w/w) silver nitrate, or include about 0.01, 0.25, 0.5, or about 1% silver nitrate, or a combination of any of the foregoing values, or a range encompassing any of the foregoing values.

In one aspect, silver can function as a preservative in the disclosed compositions. In another aspect, the inclusion of silver in the disclosed compositions can permit the exclusion of other preservatives. In a still further aspect, excluding other preservatives can present economic advantages including, but not limited to, requiring the purchase of fewer components to prepare the compositions and/or a reduction in the number of mixing steps in preparation of the compositions. In another aspect, silver can act as an active ingredient and one or more other preservatives can be present. In still another aspect, silver can function as both an active ingredient and a preservative, offering clinical efficacy as well as preservative functionality.

In one aspect, the disclosed compositions include an anti-fungal compound. In another aspect, the antifungal compound can be selected from bifonazole, clotrimazole, econazole, efinaconazole, ketoconazole, luliconazole, miconazole, sulconazole, tioconazole, terbinafine, tolciclate, tolnaftate, tavaborole, amorolfine, or any combination thereof. In one aspect, the antifungal compound is tolnaftate. In another aspect, the disclosed compositions can be useful in treating a variety of fungal infections of the skin and external surfaces of the body including, but not limited to tinea pedis (athlete's foot), tinea corporis (ringworm), tinea capitis (ringworm of the scalp), tinea cruris (fungal groin infection), tinea unguium (fungal nail infection), fungal infection of a wound, or any combination thereof.

In various aspects, the dermal health agent comprises at least one antioxidant. In a further aspect, the antioxidant comprises Vitamin A, Vitamin E, Vitamin C, Vitamin K, or combinations thereof. Antioxidants are substances which inhibit oxidation or suppress reactions promoted by oxygen or peroxides. Antioxidants, especially lipid-soluble antioxidants, can be absorbed into the cellular membrane to neutralize oxygen radicals and thereby protect the membrane. In a further aspect, the antioxidants useful in the present disclosure can be selected from the group consisting of all forms of Vitamin A (retinol), all forms of 3,4-didehydroretinol, all forms of carotene such as alpha-carotene, beta-carotene, gamma-carotene, delta-carotene, all forms of tocopherol such as Vitamin E (Alpha-tocopherol, 3,4-dihydro-2,5,7,8-tetramethyl-2-(4,8,12-trimethyltri-decyl)-2H-1-benzopyran-6-ol), beta-tocopherol, gamma-tocopherol, delta-tocopherol, tocoquinone, tocotrienol, and Vitamin E esters which readily undergo hydrolysis to Vitamin E such as Vitamin E acetate and Vitamin E succinate, and pharmaceutically acceptable Vitamin E salts such as Vitamin E phosphate, all forms of Vitamin K such as phytomenadione or phylloquinone (Vitamin K₁), phylloquinone epoxide, menaquinone (Vitamin K₂), menadione (Vitamin K₃), and Vitamin K₂ subtypes such as menaquinone-4 (MK-4), menaquinone-5 (MK-5), menaquinone-6 (MK-6), menaquinone-7 (MK-7), menaquinone-8 (MK-8), menaquinone-9 (MK-9), and menaquinone-10 (MK-10), prodrugs of Vitamin A, tretinoin (all-trans-retinoic acid, at-RA), carotene, Vitamin C, and Vitamin E, pharmaceutically acceptable salts of Vitamin A, carotene, Vitamin C, Vitamin E, and Vitamin K, and the like, and mixtures thereof. Preferably, the antioxidant is selected from the group of lipid-soluble antioxidants consisting of Vitamin A, beta-carotene, Vitamin E, Vitamin E acetate, Vitamin K, and mixtures thereof. More preferably, the antioxidant is Vitamin E or Vitamin E acetate. Most preferably, the antioxidant is Vitamin E acetate.

Without wishing to be bound by a particular theory, it is believed that Vitamin E increases the tolerance of unexposed skin to UV irradiation and supports healthy response to UV exposure. In an aspect, the effect of Vitamin E can be enhanced in combination with beta-carotene or other similar carotenoid or carotenoid derivative, e.g., Vitamin A. Additionally, without wishing to be bound by a particular theory, Vitamin E in combination with Vitamin C, or a pharmaceutically active source of Vitamin C, can protect DNA found in dermal layer cells of the skin from mutation and photodamage. In various further aspects, without wishing to be bound by a particular theory, it is believed that vitamin A can also increase fibroblast proliferation, modulate cellular differentiation and proliferation, increase collagen and hyaluronate synthesis, and decrease MMP-mediated extracellular matrix degradation.

In a further aspect, Vitamin E, or a pharmaceutically active source of Vitamin E, can comprise d-alpha tocopherol, mixed tocopherols or tocotrienols or other similarly therapeutic ingredients, alpha-tocopheryl acetate, alpha-tocopherol, and mixed tocopherols;

In a further aspect, Vitamin C can comprise a form of Vitamin C such as D-ascorbic acid, L-ascorbic acid, ascorbic acid, ester c, or other similarly therapeutic Vitamin C esters.

In a further aspect, Vitamin E is used in the present pharmaceutical compositions at a an amount, in units of vitamin E per total grams of the pharmaceutical composition, ranging from about 100 units/g to about 25000 units/g; about 200 units/g to about 25000 units/g; about 300 units/g to about 25000 units/g; about 400 units/g to about 25000 units/g; about 500 units/g to about 25000 units/g; about 600 units/g to about 25000 units/g; about 700 units/g to about 25000 units/g; about 800 units/g to about 25000 units/g; about 900 units/g to about 25000 units/g; about 1000 units/g to about 25000 units/g; about 100 units/g to about 20000 units/g; about 200 units/g to about 20000 units/g; about 300 units/g to about 20000 units/g; about 400 units/g to about 20000 units/g; about 500 units/g to about 20000 units/g; about 600 units/g to about 20000 units/g; about 700 units/g to about 20000 units/g; about 800 units/g to about 20000 units/g; about 900 units/g to about 20000 units/g; about 1000 units/g to about 20000 units/g; about 100 units/g to about 17500 units/g; about 200 units/g to about 17500 units/g; about 300 units/g to about 17500 units/g; about 400 units/g to about 17500 units/g; about 500 units/g to about 17500 units/g; about 600 units/g to about 17500 units/g; about 700 units/g to about 17500 units/g; about 800 units/g to about 17500 units/g; about 900 units/g to about 17500 units/g; about 1000 units/g to about 17500 units/g; about 100 units/g to about 15000 units/g; about 200 units/g to about 15000 units/g; about 300 units/g to about 15000 units/g; about 400 units/g to about 15000 units/g; about 500 units/g to about 15000 units/g; about 600 units/g to about 15000 units/g; about 700 units/g to about 15000 units/g; about 800 units/g to about 15000 units/g; about 900 units/g to about 15000 units/g; about 1000 units/g to about 15000 units/g; about 100 units/g to about 12500 units/g; about 200 units/g to about 12500 units/g; about 300 units/g to about 12500 units/g; about 400 units/g to about 12500 units/g; about 500 units/g to about 12500 units/g; about 600 units/g to about 12500 units/g; about 700 units/g to about 12500 units/g; about 800 units/g to about 12500 units/g; about 900 units/g to about 12500 units/g; about 1000 units/g to about 12500 units/g; about 100 units/g to about 10000 units/g; about 200 units/g to about 10000 units/g; about 300 units/g to about 10000 units/g; about 400 units/g to about 10000 units/g; about 500 units/g to about 10000 units/g; about 600 units/g to about 10000 units/g; about 700 units/g to about 10000 units/g; about 800 units/g to about 10000 units/g; about 900 units/g to about 10000 units/g; about 1000 units/g to about 10000 units/g; about 100 units/g to about 7500 units/g; about 200 units/g to about 7500 units/g; about 300 units/g to about 7500 units/g; about 400 units/g to about 7500 units/g; about 500 units/g to about 7500 units/g; about 600 units/g to about 7500 units/g; about 700 units/g to about 7500 units/g; about 800 units/g to about 7500 units/g; about 900 units/g to about 7500 units/g; about 1000 units/g to about 7500 units/g; about 100 units/g to about 5000 units/g; about 200 units/g to about 5000 units/g; about 300 units/g to about 5000 units/g; about 400 units/g to about 5000 units/g; about 500 units/g to about 5000 units/g; about 600 units/g to about 5000 units/g; about 700 units/g to about 5000 units/g; about 800 units/g to about 5000 units/g; about 900 units/g to about 5000 units/g; about 1000 units/g to about 5000 units/g; about 100 units/g to about 2500 units/g; about 200 units/g to about 2500 units/g; about 300 units/g to about 2500 units/g; about 400 units/g to about 2500 units/g; about 500 units/g to about 2500 units/g; about 600 units/g to about 2500 units/g; about 700 units/g to about 2500 units/g; about 800 units/g to about 2500 units/g; about 900 units/g to about 2500 units/g; about 1000 units/g to about 2500 units/g; about 100 units/g to about 1000 units/g; about 200 units/g to about 1000 units/g; about 300 units/g to about 1000 units/g; about 400 units/g to about 1000 units/g; about 500 units/g to about 1000 units/g; about 600 units/g to about 1000 units/g; about 700 units/g to about 1000 units/g; about 800 units/g to about 1000 units/g; about 900 units/g to about 1000 units/g; or a sub-range within the foregoing ranges; or any combination of individual values within the foregoing ranges.

In a further aspect, the Vitamin A is used in the present pharmaceutical compositions at a concentration, based on the total weight of the composition, ranging from about 0.01 to about 1 wt %; about 0.01 to about 0.9 wt %; about 0.01 to about 0.8 wt %; about 0.01 to about 0.7 wt %; about 0.01 to about 0.6 wt %; about 0.01 to about 0.5 wt %; about 0.01 to about 0.4 wt %; about 0.01 to about 0.3 wt %; about 0.01 to about 0.2 wt %; about 0.01 to about 0.1 wt %; about 0.02 to about 1 wt %; about 0.02 to about 0.9 wt %; about 0.02 to about 0.8 wt %; about 0.02 to about 0.7 wt %; about 0.02 to about 0.6 wt %; about 0.02 to about 0.5 wt %; about 0.02 to about 0.4 wt %; about 0.02 to about 0.3 wt %; about 0.02 to about 0.2 wt %; about 0.02 to about 0.1 wt %; about 0.03 to about 1 wt %; about 0.03 to about 0.9 wt %; about 0.03 to about 0.8 wt %; about 0.03 to about 0.7 wt %; about 0.03 to about 0.6 wt %; about 0.03 to about 0.5 wt %; about 0.03 to about 0.4 wt %; about 0.03 to about 0.3 wt %; about 0.03 to about 0.2 wt %; about 0.03 to about 0.1 wt %; about 0.04 to about 1 wt %; about 0.04 to about 0.9 wt %; about 0.04 to about 0.8 wt %; about 0.04 to about 0.7 wt %; about 0.04 to about 0.6 wt %; about 0.04 to about 0.5 wt %; about 0.04 to about 0.4 wt %; about 0.04 to about 0.3 wt %; about 0.04 to about 0.2 wt %; about 0.04 to about 0.1 wt %; about 0.05 to about 1 wt %; about 0.05 to about 0.9 wt %; about 0.05 to about 0.8 wt %; about 0.05 to about 0.7 wt %; about 0.05 to about 0.6 wt %; about 0.05 to about 0.5 wt %; about 0.05 to about 0.4 wt %; about 0.05 to about 0.3 wt %; about 0.05 to about 0.2 wt %; about 0.05 to about 0.1 wt %; about 0.06 to about 1 wt %; about 0.06 to about 0.9 wt %; about 0.06 to about 0.8 wt %; about 0.06 to about 0.7 wt %; about 0.06 to about 0.6 wt %; about 0.06 to about 0.5 wt %; about 0.06 to about 0.4 wt %; about 0.06 to about 0.3 wt %; about 0.06 to about 0.2 wt %; about 0.06 to about 0.1 wt %; or a sub-range within the foregoing ranges; or any combination of individual values within the foregoing ranges.

In a further aspect, the Vitamin C is used in the present pharmaceutical compositions at a concentration, based on the total weight of the composition, ranging from about 0.1 to about 20 wt %; about 0.1 to about 15 wt %; about 0.1 to about 10 wt %; about 0.1 to about 7 wt %; about 0.1 to about 5 wt %; about 0.1 to about 4 wt %; about 0.1 to about 3 wt %; about 0.1 to about 2 wt %; about 0.1 to about 1 wt %; about 0.2 to about 20 wt %; about 0.2 to about 15 wt %; about 0.2 to about 10 wt %; about 0.2 to about 10 wt %; about 0.2 to about 7 wt %; about 0.2 to about 5 wt %; about 0.2 to about 4 wt %; about 0.2 to about 3 wt %; about 0.2 to about 2 wt %; about 0.2 to about 1 wt %; about 0.3 to about 20 wt %; about 0.3 to about 15 wt %; about 0.3 to about 10 wt %; about 0.3 to about 10 wt %; about 0.3 to about 7 wt %; about 0.3 to about 5 wt %; about 0.3 to about 4 wt %; about 0.3 to about 3 wt %; about 0.3 to about 2 wt %; about 0.3 to about 1 wt %; about 0.14 to about 20 wt %; about 0.4 to about 15 wt %; about 0.4 to about 10 wt %; about 0.4 to about 10 wt %; about 0.4 to about 7 wt %; about 0.4 to about 5 wt %; about 0.4 to about 4 wt %; about 0.4 to about 3 wt %; about 0.4 to about 2 wt %; about 0.4 to about 1 wt %; about 0.5 to about 20 wt %; about 0.5 to about 15 wt %; about 0.5 to about 10 wt %; about 0.5 to about 10 wt %; about 0.5 to about 7 wt %; about 0.5 to about 5 wt %; about 0.5 to about 4 wt %; about 0.5 to about 3 wt %; about 0.5 to about 2 wt %; about 0.5 to about 1 wt %; about 1 to about 20 wt %; about 1 to about 15 wt %; about 1 to about 10 wt %; about 1 to about 10 wt %; about 1 to about 7 wt %; about 1 to about 5 wt %; about 1 to about 4 wt %; about 1 to about 3 wt %; about 1 to about 2 wt %; about 5 to about 20 wt %; about 5 to about 15 wt %; about 5 to about 10 wt %; about 5 to about 10 wt %; about 5 to about 7 wt %; or a sub-range within the foregoing ranges; or any combination of individual values within the foregoing ranges.

In a further aspect, the Vitamin K is used in the present pharmaceutical compositions at a concentration, based on the total weight of the composition, ranging from about 100 μg to about 50 mg; about 100 μg to about 25 mg, about 100 μg to about 2.5 mg; about 100 μg to about 1 mg; about 100 μg to about 900 μg, about 100 μg to about 800 μg; about 100 μg to about 700 μg; about 100 μg to about 600 μg; about 100 μg to about 500 μg, about 100 μg to about 500 μg; about 100 μg to about 400 μg, about 100 μg to about 300 μg, about 100 μg to about 200 μg; about 2.5 mg to about 25 mg, about 2.5 mg to about 10 mg, about 5 mg to about 25 mg, about 5 mg to about 24 mg; about 5 mg to about 23 mg; about 5 mg to about 22 mg; about 5 mg to about 21 mg; about 5 mg to about 20 mg; about 5 mg to about 19 mg; about 5 mg to about 18 mg; about 5 mg to about 17 mg; about 5 mg to about 16 mg; about 5 mg to about 15 mg; about 5 mg to about 14 mg; about 5 mg to about 13 mg; about 5 mg to about 12 mg; about 5 mg to about 11 mg; about 5 mg to about 10 mg; about 2.5 mg to about 9 mg; about 3 mg to about 9 mg; about 3 mg to about 8 mg; about 3 mg to about 7 mg; about 3 mg to about 6 mg; about 3 mg to about 5 mg; about mg to about 4 mg; about 3 mg to about 3.5 mg; about 2.5 mg to about 3 mg; about 2.5 mg to about 2.9 mg; about 2.5 mg to about 2.8 mg; about 2.5 mg to about 2.7 mg; about 2.5 mg to about 2.6 mg; or a sub-range within the foregoing ranges; or any combination of individual values within the foregoing ranges.

In various aspects, the therapeutic agent can further comprise at least one form of Vitamin D, or a pharmaceutically active source of Vitamin D.

In various aspects, the therapeutic agent can further comprise at least one steroidal agent, e.g., a corticosteroid such as hydrocortisone.

### Secondary Agents

In various aspects, the disclosed pharmaceutical compositions can further comprise a secondary agent. In a further aspect, the secondary agent can comprise at least one agent selected from calendula oil, cucumber oil, witch hazel oil or extract, lavender oil, olive oil, coconut oil, capryl caprylate triglyceride, shea butter, D-panthenol, glycerine, allantoin, oatmeal extract, and combinations thereof. USP grade versions or compatible for external use in humans of each of the foregoing are commercially available. When present in the disclosed pharmaceutical compositions, the foregoing can be present in the amounts provided below in Table 1.

TABLE 1
Secondary Agents

| Ingredient | Quantity % w/w |
|---|---|
| Sodium Hyaluronate | 0.2-1.0 |
| Calendula Oil | 0.5-2.0 |
| Cucumber oil | 0.5-2.0 |
| Witch hazel extract | 2.0-5.0 |
| Lavender oil | 0.5-2.0 |
| Olive oil | 0.5-2.0 |
| Coconut oil/Capryl caprylate | 2.0-4.0 |
| triglyceride | |
| D-Panthenol | 0.5-1.0 |
| Shea butter | 1.0-2.0 |
| Glycerine | 3.0-5.0 |
| Allantoin | 0.2-0.4 |
| Oatmeal extract | 0.5-1.0 |

### Pharmaceutical Compositions

In various aspects, the present disclosure relates to pharmaceutical compositions comprising a therapeutically effective amount of at least one disclosed therapeutic agent, or a pharmaceutically acceptable salt, a hydrate thereof, a solvate thereof, a polymorph thereof, in which the pharmaceutical composition is useful for topical treatment of a burn, including, but not limited to, sunburns, 3^{rd} degree burns, and the like. It is understood that the therapeutic agent can comprise one or more therapeutic agents. That is, the therapeutic agent comprises a plurality of therapeutic agents. In some instances, the therapeutic agent comprises a single therapeutic agent. The disclosed pharmaceutical compositions can further comprise other therapeutically active compounds, which can be useful in the treatment of a burn, e.g., a sunburn. It is understood that the disclosed pharmaceutical compositions can be prepared from the disclosed therapeutic agents. It is also understood that the disclosed compositions can be employed in the disclosed methods of using.

In a further aspect, the present disclosure relates to a pharmaceutical composition comprising a therapeutically effective amount of a disclosed therapeutic agent, a pharmaceutically acceptable salt, a hydrate thereof, a solvate thereof, a polymorph thereof, and a pharmaceutically acceptable carrier. Additionally, the present disclosure relates to a process for preparing such a pharmaceutical composition, characterized in that a pharmaceutically acceptable carrier is intimately mixed with a therapeutically effective amount of a compound according to the present disclosure.

In a further aspect, it is understood that a therapeutic agent can comprise a first therapeutic agent, a second therapeutic agent, a fourth therapeutic agent, and a fifth therapeutic agent, or a pharmaceutically acceptable salt thereof each therapeutic agent. In a still further aspect, the first therapeutic agent comprises a dermal restorative agent, the second therapeutic agent comprises a pain modulator agent, the third therapeutic agent comprises soothing agent, the fourth therapeutic agent comprises an anti-inflammatory agent, and the fifth therapeutic agent comprises a dermal health agent. Moreover, it is further understood that each of the therapeutic agents, such as the first therapeutic agent, the second therapeutic agent, the fourth therapeutic agent, and the fifth therapeutic agent, can comprise one or more first therapeutic agent, one or more second therapeutic agent, one or more fourth therapeutic agent, and one or more fifth therapeutic agent. That is, each of the therapeutic agents, such as the first therapeutic agent, the second therapeutic agent, the fourth therapeutic agent, and the fifth therapeutic agent, can comprise a plurality of first therapeutic agents, a plurality of therapeutic agents, a plurality of fourth therapeutic agents, and a plurality of fifth therapeutic agents. In some instances, the first therapeutic agent, the second therapeutic agent, the fourth therapeutic agent, and the fifth therapeutic agent each comprise a single therapeutic agent.

In a further aspect, the present disclosure relates to pharmaceutical compositions comprising at least one disclosed secondary agent. In one aspect, the secondary agent can be sodium hyaluronate. Further in this aspect, the concentration of sodium hyaluronate can impact the viscosity and/or stickiness of the compositions. In one aspect, the concentration of sodium hyaluronate can be from about 0.01% by weight to about 3% by weight, or can be about 0.01, 0.05, 0.1, 0.5, 1, 1.5, 2, 2.5, or about 3% by weight, or a combination of any of the foregoing values, or a range encompassing any of the foregoing values. In some aspects, then the concentration of sodium hyaluronate is high, the compositions can be sticky and viscous. In other aspects, when the concentration of sodium hyaluronate is lower, the compositions can be less viscous. Further in this aspect, compositions that are less viscous may be smoother to the touch, less sticky, and/or easier to rub into the skin. In some aspects, stickiness can be reduced or offset by increasing the concentration of another secondary agent such as, for example, an oil.

In a further aspect, the present disclosure relates to pharmaceutical compositions comprising a therapeutically effective amount of a first therapeutic agent, a second therapeutic agent, a fourth therapeutic agent, and a fifth therapeutic agent, or a pharmaceutically acceptable salt thereof each therapeutic agent, such that the first therapeutic agent is dermal restorative agent, the second therapeutic agent is a pain modulator agent, the third therapeutic agent is soothing agent, the fourth therapeutic agent is an anti-inflammatory agent, and the fifth therapeutic agent is a dermal health agent, in which the pharmaceutical composition is useful for topical treatment of a burn, including, but not limited to, sunburns, 3^{rd} degree burns, and the like.

As used herein, “pharmaceutically-acceptable carriers” means one or more of a pharmaceutically acceptable diluents, preservatives, antioxidants, solubilizers, emulsifiers, coloring agents, releasing agents, coating agents, sweetening, flavoring and perfuming agents, and adjuvants. The disclosed pharmaceutical compositions can be conveniently presented in unit dosage form and prepared by any of the methods well known in the art of pharmacy and pharmaceutical sciences.

The disclosed pharmaceutical compositions of the present disclosure therefore not only have the effects attributable to the individual ingredients, but above all have a positive effect for treatment of burns, particularly sunburn, including accelerating healing, reducing pain and discomfort, and providing a positive user experience due to a synergistic effect of the first therapeutic agent comprising a disclosed dermal restorative agent, the second therapeutic agent comprising a disclosed pain modulator agent, the third therapeutic agent comprising a disclosed soothing agent, the fourth therapeutic agent comprising a disclosed anti-inflammatory agent, and the fifth therapeutic agent comprising a disclosed dermal health agent in the amounts provided above in the discussion of therapeutic agents.

The disclosed pharmaceutical compositions of the present disclosure therefore not only have the effects attributable to the individual ingredients, but above all have a positive effect for treatment of burns, particularly sunburn, including accelerating healing, reducing pain and discomfort, and providing a positive user experience due to a synergistic effect of the first therapeutic agent comprising a disclosed dermal restorative agent, the second therapeutic agent comprising a disclosed pain modulator agent, the third therapeutic agent comprising a disclosed soothing agent, the fourth therapeutic agent comprising a disclosed anti-inflammatory agent, and the fifth therapeutic agent comprising a disclosed dermal health agent in the amounts provided above in the discussion of therapeutic agents, and further comprising a secondary agent as disclosed herein above at the disclosed amounts.

In various aspects, the disclosed pharmaceutical composition comprises a first therapeutic agent comprising dermal restorative agent present in amount as disclosed herein above, e.g., at a concentration ranging from 0.1 to 4% by weight of the total weight of the composition, preferably from 0.2 to 2%.

In a further, the disclosed pharmaceutical composition comprises a second therapeutic agent comprising a pain modulator agent present in amount as disclosed herein above, e.g., lidocaine hydrochloride at a concentration of from about 0.1 wt % to 5 wt %.

In a further aspect, the disclosed pharmaceutical composition comprises a third therapeutic agent comprising a soothing agent present in amount as disclosed herein above, e.g., aloe and/or derivative present in an amount of about 0 wt %-50 wt %.

In a further aspect, the disclosed pharmaceutical composition comprises a fourth therapeutic agent comprising an anti-inflammatory agent, i.e., a disclosed NSAID, present in amount as disclosed herein above, e.g., diclofenac in acid form at a concentration ranging from 0.4 to 3 wt % of the total weight of the composition, or about 0.6 to 2.4 wt %, or about from 0.8 to 1.2%; or alternatively ketoprofen, in the amount of 1 wt % to 15 wt %, including, but not limited to, 1 wt % to 5 wt %, 4 wt % to 9 wt %, 8 wt % to 12 wt %, or 11 wt % to 15 wt %, based on the weight of the composition

In a further aspect, the disclosed pharmaceutical composition comprises a fourth therapeutic agent comprising an antioxidant, such as vitamin A and vitamin E, present in amount as disclosed herein above, e.g., Vitamin E in the form of a tocopherol present in an amount from about 100 units/g to about 2500 units/g of the pharmaceutical composition. The exact amount of antioxidant is a matter of preference subject to such factors as the type of condition being treated as well as the other ingredients in the composition. In a preferred aspect, the antioxidant is present in the therapeutic wound healing composition in an amount from about 0.1% to about 40%, preferably from about 0.2% to about 30%.

Depending on the mode of administration, the disclosed pharmaceutical composition can comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

In a further aspect, the disclosed pharmaceutical compositions comprise a therapeutically effective amount of at least one therapeutic agent, or a pharmaceutically acceptable salt thereof as an active ingredient, a pharmaceutically acceptable carrier, optionally one or more other therapeutic agent, and optionally one or more adjuvant. The disclosed pharmaceutical compositions include those suitable for topical administration.

In various aspects, the present disclosure also relates to a pharmaceutical composition comprising a pharmaceutically acceptable carrier or diluent and a therapeutically effective amount of a disclosed therapeutic agent, a pharmaceutically acceptable salt, a hydrate thereof, a solvate thereof, a polymorph thereof, or a stereochemically isomeric form thereof.

Pharmaceutically acceptable salts can be prepared from pharmaceutically acceptable non-toxic bases or acids. For therapeutic use, salts of the disclosed compounds are those wherein the counter ion is pharmaceutically acceptable. However, salts of acids and bases which are non-pharmaceutically acceptable may also find use, for example, in the preparation or purification of a pharmaceutically acceptable compound. All salts, whether pharmaceutically acceptable or not, are contemplated by the present disclosure. Pharmaceutically acceptable acid and base addition salts are meant to comprise the therapeutically active non-toxic acid and base addition salt forms which the disclosed compounds are able to form.

In various aspects, a disclosed compound comprising an acidic group or moiety, e.g., a carboxylic acid group, can be used to prepare a pharmaceutically acceptable salt. For example, such a disclosed compound may comprise an isolation step comprising treatment with a suitable inorganic or organic base. In some cases, it may be desirable in practice to initially isolate a compound from the reaction mixture as a pharmaceutically unacceptable salt and then simply convert the latter back to the free acid compound by treatment with an acidic reagent, and subsequently convert the free acid to a pharmaceutically acceptable base addition salt. These base addition salts can be readily prepared using conventional techniques, e.g., by treating the corresponding acidic compounds with an aqueous solution containing the desired pharmacologically acceptable cations and then evaporating the resulting solution to dryness, preferably under reduced pressure. Alternatively, they also can be prepared by mixing lower alkanolic solutions of the acidic compounds and the desired alkali metal alkoxide together, and then evaporating the resulting solution to dryness in the same manner as before.

Bases which can be used to prepare the pharmaceutically acceptable base-addition salts of the base compounds are those which can form non-toxic base-addition salts, i.e., salts containing pharmacologically acceptable cations such as, alkali metal cations (e.g., lithium, potassium and sodium), alkaline earth metal cations (e.g., calcium and magnesium), ammonium or other water-soluble amine addition salts such as N-methylglucamine-(meglumine), lower alkanolammonium and other such bases of organic amines. In a further aspect, derived from pharmaceutically acceptable organic non-toxic bases include primary, secondary, and tertiary amines, as well as cyclic amines and substituted amines such as naturally occurring and synthesized substituted amines. In various aspects, such pharmaceutically acceptable organic non-toxic bases include, but are not limited to, ammonia, methylamine, ethylamine, propylamine, isopropylamine, any of the four butylamine isomers, betaine, caffeine, choline, dimethylamine, diethylamine, diethanolamine, dipropylamine, diisopropylamine, di-n-butylamine, N,N′-dibenzylethylenediamine, pyrrolidine, piperidine, morpholine, trimethylamine, triethylamine, tripropylamine, tromethamine, 2-diethylaminoethanol, 2-dimethylaminoethanol, ethanolamine, quinuclidine, pyridine, quinoline and isoquinoline; benzathine, N-methyl-D-glucamine, ethylenediamine, N-ethylmorpholine, N-ethylpiperidine, glucamine, glucosamine, methylglucamine, morpholine, piperazine, piperidine, polyamine resins, procaine, purines, theobromine, hydrabamine salts, and salts with amino acids such as, for example, histidine, arginine, lysine and the like. The foregoing salt forms can be converted by treatment with acid back into the free acid form.

In various aspects, a disclosed therapeutic agent comprising a protonatable group or moiety, e.g., an amino group, can be used to prepare a pharmaceutically acceptable salt. For example, such a disclosed therapeutic agent may comprise an isolation step comprising treatment with a suitable inorganic or organic acid. In some cases, it may be desirable in practice to initially isolate a compound from the reaction mixture as a pharmaceutically unacceptable salt and then simply convert the latter back to the free base compound by treatment with a basic reagent, and subsequently convert the free base to a pharmaceutically acceptable acid addition salt. These acid addition salts can be readily prepared using conventional techniques, e.g., by treating the corresponding basic compounds with an aqueous solution containing the desired pharmacologically acceptable anions and then evaporating the resulting solution to dryness, preferably under reduced pressure. Alternatively, they also can be prepared by treating the free base form of the disclosed compound with a suitable pharmaceutically acceptable non-toxic inorganic or organic acid.

Acids which can be used to prepare the pharmaceutically acceptable acid-addition salts of the base compounds are those which can form non-toxic acid-addition salts, i.e., salts containing pharmacologically acceptable anions formed from their corresponding inorganic and organic acids. Exemplary, but non-limiting, inorganic acids include hydrochloric hydrobromic, sulfuric, nitric, phosphoric and the like. Exemplary, but non-limiting, organic acids include acetic, benzenesulfonic, benzoic, camphorsulfonic, citric, ethanesulfonic, fumaric, gluconic, glutamic, isethionic, lactic, maleic, malic, mandelicmethanesulfonic, mucic, pamoic, pantothenic, succinic, tartaric, p-toluenesulfonic acid and the like. In a further aspect, the acid-addition salt comprises an anion formed from hydrobromic, hydrochloric, maleic, phosphoric, sulfuric, and tartaric acids.

In practice, the therapeutic agent of the present disclosure, or pharmaceutically acceptable salts thereof, of the present disclosure can be combined in intimate admixture with a pharmaceutical carrier according to conventional pharmaceutical compounding techniques.

Techniques and compositions for making dosage forms useful for materials and methods described herein are described, for example, in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

The therapeutic agent described herein are typically to be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable compound will be in a form suitable for oral, rectal, topical, intravenous injection or parenteral administration. Carriers include solids or liquids, and the type of carrier is chosen based on the type of administration being used. The compounds may be administered as a dosage that has a known quantity of the compound.

In some instances, a disclosed topical formulation can be in the form of a solution or suspension. In such instances, it is, for example, possible to use water, particularly sterile water, or physiologically acceptable organic solvents, such as alcohols (ethanol, propanol, isopropanol, 1,2-propylene glycol, polyglycols and their derivatives, fatty alcohols, partial esters of glycerol), oils (for example peanut oil, olive oil, sesame oil, almond oil, sunflower oil, soya bean oil, castor oil, bovine hoof oil), paraffins, dimethyl sulfoxide, triglycerides and the like.

The pharmaceutical compositions of the present disclosure can optionally contain at least one antimicrobial preservative in the range of 0.001% to about 0.3% by weight/volume of the composition. Examples of such antimicrobial preservatives are benzalkonium chloride, cetylpyridinium chloride/bromide, chlorobutanol, chlorhexidine acetate, chlorhexidine HCl, chlorhexidine digluconate, chlorocresol, methylparaben, propylparaben, butylparaben, phenoxyethanol, phenylmercury salts, sorbic acid, thiomersal. A preferred preservative which functions as an antimicrobial agent includes the commercially available preservative, benzalkonium chloride in the range of about 0.02 to about 0.025% by weight/volume. In addition to being preservatives, stronger antibiotics may be added at therapeutic levels well known in the art, if treatment of a patient with an antibiotic is indicated.

In one aspect, the preservative can include high purity monoesters of caprylic acid and undecylenic acid. In a further aspect, the preservative can possess biostatic activity against bacteria and yeast as well as, in some aspects, against other fungi. In yet another aspect, the preservative can further act as an emollient, co-emulsifier, skin re-fatting agent, or any combination thereof. In still another aspect, the preservative can be stable and effective at from about pH 4.0 to about pH 8.0, or at about pH 4.0, 4.5, 5.0, 5.5, 6.0, 6.5, 7.0, 7.5, or about 8.0. In one aspect, the preservative may function optimally at about pH 5.5 or lower. In any of these aspects, the preservative can be LEXGARD® Natural MB (Inolex, Philadelphia, Pa.) or a similar or related compound.

It may optionally be necessary to stabilize a disclosed pharmaceutical composition, e.g., a disclosed topical formulation. In a further aspect, the pharmaceutical compositions of the present disclosure can also optionally include pharmaceutically acceptable buffers sufficient to adjust and maintain the pH of the compositions of the present disclosure in the range of about 4.0 to about 9.0, preferably about 5.5 to about 7.0 and 6.25 to 6.75 being most preferable. Typically suitable buffers include citrate, phosphate, tromethamine, glycine, borate or acetate salts, which can also be derived from substances of the type such as citric acid, primary or secondary sodium phosphate, glycine, boric acid, sodium tetraborate, acetic acid and sodium acetate. Further excipients such as hydrochloric acid or sodium hydroxide can also be used for pH adjustment.

In some aspects, stabilizing and texture-modifying components can be included in the disclosed compositions. In one aspect, hydroxyethyl acrylate/sodium acryloyldimethyl taurate copolymer can provide useful thickening, stabilizing, and texturizing functions. In another aspect, the stabilizing and/or texture-modifying component can be provided in the form of a polymer powder suitable for use from pH 3 to pH 12, or at pH 3, 3.5, 4, 4.5, 5, 5.5, 6, 6.5, 7, 7.5, 8, 8.5, 9, 9.5, 10, 10.5, 11, 11.5, or 12, or a combination of any of the foregoing values, or a range encompassing any of the foregoing values. In one aspect, the stabilizing and/or texture-modifying component can provide a smooth and soft texture that is easy for the consumer to apply. In another aspect, the stabilizing and/or texture-modifying component is a non-microplastic polymer and is biodegradable, and is thus environmentally benign. In one aspect, the stabilizing and/or texture-modifying component can be SEPINOV™ EMT 10 (Seppic SA, La Garenne-Colombes, France), or a similar or related compound.

In order to enhance the solubility and/or the stability of a disclosed therapeutic agent in a disclosed liquid dosage form, a parenteral injection form, or an intravenous injectable form, it can be advantageous to employ α-, β- or γ-cyclodextrins or their derivatives, in particular hydroxyalkyl substituted cyclodextrins, e.g. 2-hydroxypropyl-β-cyclodextrin or sulfobutyl-β-cyclodextrin. Also co-solvents such as alcohols may improve the solubility and/or the stability of the compounds according to the present disclosure in pharmaceutical compositions.

In various aspects, a disclosed pharmaceutical composition, e.g., a topical formulation, can further comprise liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

In a particular aspect, the pharmaceutical compositions of the present disclosure can be in a form suitable for topical administration. In various aspects, the topical composition may include a colloid or emulsion (o/w, w/o), cream, lotion, ointment, foam, aqueous or non-aqueous gel, aqueous or non-aqueous solution, which may include a dispersion, powder, nail lacquer, bath, or paste. As used herein, the phrase “topical application” means administration onto a biological surface, whereby the biological surface includes, for example, a skin area (e.g., hands, forearms, elbows, legs, face, nails, anus and genital areas) or a mucosal membrane. By selecting the appropriate carrier and optionally other ingredients that can be included in the composition, as is detailed herein below, the compositions of the present disclosure may be formulated into any form typically employed for topical application. A topical pharmaceutical composition can be in a form of a cream, an ointment, a paste, a gel, a lotion, milk, a suspension, an aerosol, a spray, foam, a dusting powder, a pad, and a patch. Further, the compositions can be in a form suitable for use in transdermal devices. These formulations can be prepared, utilizing a compound of the present disclosure, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Ointments are semisolid preparations, typically based on petrolatum or petroleum derivatives. The specific ointment base to be used is one that provides for optimum delivery for the active agent chosen for a given formulation, and, preferably, provides for other desired characteristics as well (e.g., emollience). As with other carriers or vehicles, an ointment base should be inert, stable, nonirritating and nonsensitizing. As explained in Remington: The Science and Practice of Pharmacy, 19th Ed., Easton, Pa.: Mack Publishing Co. (1995), pp. 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight.

Lotions are preparations that are to be applied to the skin surface without friction. Lotions are typically liquid or semiliquid preparations in which solid particles, including the active agent, are present in a water or alcohol base. Lotions are typically preferred for treating large body areas, due to the ease of applying a more fluid composition. Lotions are typically suspensions of solids, and oftentimes comprise a liquid oily emulsion of the oil-in-water type. It is generally necessary that the insoluble matter in a lotion be finely divided. Lotions typically contain suspending agents to produce better dispersions as well as compounds useful for localizing and holding the active agent in contact with the skin, such as methylcellulose, sodium carboxymethyl-cellulose, and the like.

Creams are viscous liquids or semisolid emulsions, either oil-in-water or water-in-oil. Cream bases are typically water-washable, and contain an oil phase, an emulsifier and an aqueous phase. The oil phase, also called the “internal” phase, is generally comprised of petrolatum and/or a fatty alcohol such as cetyl or stearyl alcohol. The aqueous phase typically, although not necessarily, exceeds the oil phase in volume, and generally contains a humectant. The emulsifier in a cream formulation is generally a nonionic, anionic, cationic or amphoteric surfactant. Reference may be made to Remington: The Science and Practice of Pharmacy, supra, for further information.

Pastes are semisolid dosage forms in which the bioactive agent is suspended in a suitable base. Depending on the nature of the base, pastes are divided between fatty pastes or those made from a single-phase aqueous gel. The base in a fatty paste is generally petrolatum, hydrophilic petrolatum and the like. The pastes made from single-phase aqueous gels generally incorporate carboxymethylcellulose or the like as a base. Additional reference may be made to Remington: The Science and Practice of Pharmacy, for further information.

Gel formulations are semisolid, suspension-type systems. Single-phase gels contain organic macromolecules distributed substantially uniformly throughout the carrier liquid, which is typically aqueous, but also, preferably, contain an alcohol and, optionally, an oil. Preferred organic macromolecules, i.e., gelling agents, are crosslinked acrylic acid polymers such as the family of carbomer polymers, e.g., carboxypolyalkylenes that may be obtained commercially under the trademark Carbopol™. Other types of preferred polymers in this context are hydrophilic polymers such as polyethylene oxides, polyoxyethylene-polyoxypropylene copolymers and polyvinylalcohol; modified cellulose, such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Sprays generally provide the active agent in an aqueous and/or alcoholic solution which can be misted onto the skin for delivery. Such sprays include those formulated to provide for concentration of the active agent solution at the site of administration following delivery, e.g., the spray solution can be primarily composed of alcohol or other like volatile liquid in which the active agent can be dissolved. Upon delivery to the skin, the carrier evaporates, leaving concentrated active agent at the site of administration.

Foam compositions are typically formulated in a single or multiple phase liquid form and housed in a suitable container, optionally together with a propellant which facilitates the expulsion of the composition from the container, thus transforming it into a foam upon application. Other foam forming techniques include, for example the “Bag-in-a-can” formulation technique. Compositions thus formulated typically contain a low-boiling hydrocarbon, e.g., isopropane. Application and agitation of such a composition at the body temperature cause the isopropane to vaporize and generate the foam, in a manner similar to a pressurized aerosol foaming system. Foams can be water-based or aqueous alkanolic, but are typically formulated with high alcohol content which, upon application to the skin of a user, quickly evaporates, driving the active ingredient through the upper skin layers to the site of treatment.

Examples of pharmaceutically acceptable carriers that are suitable for pharmaceutical compositions for topical applications include carrier materials that are well-known for use in the cosmetic and medical arts as bases for e.g., emulsions, creams, aqueous solutions, oils, ointments, pastes, gels, lotions, milks, foams, suspensions, aerosols and the like, depending on the final form of the composition. Representative examples of suitable carriers according to the present disclosure therefore include, without limitation, water, liquid alcohols, liquid glycols, liquid polyalkylene glycols, liquid esters, liquid amides, liquid protein hydrolysates, liquid alkylated protein hydrolysates, liquid lanolin and lanolin derivatives, and like materials commonly employed in cosmetic and medicinal compositions. Other suitable carriers according to the present disclosure include, without limitation, alcohols, such as, for example, monohydric and polyhydric alcohols, e.g., ethanol, isopropanol, glycerol, sorbitol, 2-methoxyethanol, diethyleneglycol, ethylene glycol, hexyleneglycol, mannitol, and propylene glycol; ethers such as diethyl or dipropyl ether; polyethylene glycols and methoxypolyoxyethylenes (carbowaxes having molecular weight ranging from 200 to 20,000); polyoxyethylene glycerols, polyoxyethylene sorbitols, stearoyl diacetin, and the like.

In various aspects, a disclosed topical composition may include a carrier comprising one or more carriers or carrier components thereof.

The carrier may be liquid, semi-liquid, or solid. For example, the carrier may include an aqueous, organic, or inorganic solution, which may include a dispersion or suspension, cream, gel, ointment, lotion, emulsion, powder, or paste. In an aspect, the carrier includes a carrier or vehicle composition such as a base cream, ointment, gel, lotion, foam, or solution. The carrier may include carrier components such as lecithin, phospholipids, glycols, paraffin, fatty acids, carbopols/carbomers, alcohols, lanolin, for example.

In a further aspect, the carrier comprises an aqueous solution. In some examples, the carriers comprising aqueous solutions may be combined with the antimicrobial agent to formulate a topical composition comprising an irrigation solution, a footbath, a nail lacquer, a topical spray or soak, for example. In an aspect, the carrier or component may include an aqueous solution comprising a saline solution. For example, the topical composition may comprise a carrier or component comprising a sodium hydroxide solution, which may be a sterile solution, an alcohol, water, e.g., purified water, water for irrigation, water for injection, or a sterile water. In one aspect, a carrier or component comprises a sodium chloride 0.09% solution (sterile). The carrier or component may be present in an amount sufficient to obtain the desired amount of active agents per unit weight or volume.

In a further aspect, the topical composition may include a carrier comprising a polyethylene glycol (PEG) carrier component. In other aspects, the composition is PEG-free. In these or other aspects, the composition may include a silicon or silicon variant carrier component. In an aspect, the composition is silicon-free. An example topical composition may comprise a solution including carrier components selected from water, alcohol, DMSO, saline or sodium chloride, sodium hypochlorite, or other aqueous or non-aqueous carrier medium into which the one or more active agents are mixed, dispersed, solubilized, or dissolved. The topical composition may be water soluble/miscible or formulated for water absorption. The topical composition may comprise a water-in-oil emulsion or oil-in-water emulsion. In one aspect, the topical composition comprises a emulsion, e.g., a cream or lotion format, comprising one or more carrier components selected from of acrylate copolymer, alcohol, camphor, carbomer, dimethyl isosorbide, disodium EDTA, dl-alphatocopheryl acetate, edetate disodium, emulsifying wax, eucalyptus oil, flavonoids, glycerin, glycol dicaprylate/dicaprate, hydroxyethyl cellulose, isopropyl myristate, lactic acid, meadowsweet extract, menthol, mineral oil, neopentyl, phenolic glycosides, polyethylene glycol (PEG), polysorbate (e.g., polysorbate 85, polysorbate 20), purified water, titanium dioxide, tridecyl stearate, tridecyl trimellitate, sodium hydroxide, sodium hydroxide, sorbitol, stearic acid, zinc pyrithione, or combinations thereof. In an aspect, the topical composition comprises a foam format that includes propellant carrier component such as butane. Topical compositions comprising a foam format may also comprise additional characteristics such as that of an emulsion, such as an oil-in-water emulsion, or gel.

In a further aspect, the topical composition comprises an ointment format and includes active agents in a carrier comprising carrier components selected from hydrophilic petrolatum, white petrolatum, hydrophilic ointment, white ointment, anhydrous lanolin, hydrous lanolin, PEG ointment, or combinations thereof. In an aspect, the topical composition comprises a gel format. The gel may be an aqueous or non-aqueous gel. The gel may include carrier components thickening agents and/or gelling agents such as carbopol, poloxamer, xanthan gum, methylcellulose, carboxymethyl cellulose, hydroxyethyl cellulose, ethylcellulose, gelatin, magnesium aluminum silicate, polyvinyl alcohol, sodium alginate, or combinations thereof. The topical composition may include a powder format and include carrier components such as lactose or talc, for example.

In a further aspect, the topical composition or carrier thereof may include carrier components such as one or more solubilizers, stabilizers, buffers, tonicity modifiers, bulking agents, viscosity enhancers/reducers, surfactants, chelating agents, adjuvants, or combinations thereof.

In various aspects, the topical composition or carrier thereof comprises one or more glucose polymers such as a starch, cellulose, polydextrose, or combination thereof. Example starches may include sodium starch glycolate, corn starch, pregelatinized starch, or combination thereof. Example celluloses may include hydroxypropyl cellulose, hypermellose, croscarmellose sodium, ethyl cellulose, microcrystalline cellulose, or combination thereof. Povidone such as povidone K30, copovidone, crospovidone, or combination thereof, may also be present. In an aspect, glycol and/or a sugar alcohol may be present. Example glycols may include polyethylene glycol, propylene glycol, or combination thereof. Example sugar alcohols may include mannitol. Some aspects may include oxides such as silicon dioxide, titanium dioxide, ferric oxide, or combination thereof. One aspect may include any of the above and magnesium stearate, talc, diethyl phthalate, sodium stearyl fumarate, sodium lauryl sulfate, polysorbate, triacetin, polacrilin, lactose, glycerol behenate, polyvinyl alcohol, carnauba wax, or combination thereof. In one aspect, the topical composition does not include one or more of starch, cellulose, polydextrose, sodium starch glycolate, corn starch, pregelatinized starch, hydroxypropyl cellulose, hypermellose, croscarmellose sodium, ethyl cellulose, microcrystalline cellulose, povidone, povidone K30, copovidone, crospovidone, polyethylene glycol, propylene glycol, mannitol, silicon dioxide, titanium dioxide, ferric oxide, magnesium stearate, talc, diethyl phthalate, sodium stearyl fumarate, sodium lauryl sulfate, polysorbate, triacetin, polacrilin, lactose, glycerol behenate, polyvinyl alcohol, carnauba wax, or combination thereof.

In a further aspect, the topical composition may be administered topically by contacting an external surface of the body. The topical composition may be administered in a spray, coat, soak, powder, spread, or the like, for example, suitable to the topical format.

In a further aspect, the topical compositions of the present disclosure can, if desired, be presented in a pack or dispenser device, such as an FDA-approved kit, which may contain one or more unit dosage forms containing the active ingredient. The dispenser device may, for example, comprise a tube. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising the topical composition of the disclosure formulated in a pharmaceutically acceptable carrier may also be prepared, placed in an appropriate container, and labeled for treatment of an indicated condition.

Pharmaceutical compositions containing a therapeutic agent of the present disclosure, and/or pharmaceutically acceptable salts thereof, can also be prepared in powder or liquid concentrate form that can subsequently be used to prepare a topical formulation for use by a subject or patient. It is contemplated that such powder or liquid concentrates could be provided to a compounding pharmacy, in-patient clinic, out-patient clinic, or hospital to utilize in the preparation of a topical formulation for use with subjects or patients.

The disclosed pharmaceutical compositions (or formulation) may be packaged in a variety of ways. Generally, an article for distribution includes a container that contains the pharmaceutical composition in an appropriate form. Suitable containers are well known to those skilled in the art and include materials such as bottles (plastic and glass), sachets, foil packs, and the like. The container may also include a tamper proof assemblage to prevent indiscreet access to the contents of the package. In addition, the container typically has deposited thereon a label that describes the contents of the container and any appropriate warnings or instructions.

The disclosed pharmaceutical compositions may, if desired, be presented in a pack or dispenser device which may contain one or more unit dosage forms containing the active ingredient. The pack may for example comprise metal or plastic foil pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Pharmaceutical compositions comprising a disclosed compound formulated in a compatible pharmaceutical carrier may also be prepared, placed in an appropriate container, and labeled for treatment of an indicated condition.

The exact dosage and frequency of administration depends on the particular disclosed therapeutic agent, and amounts thereof; the particular condition being treated and the severity of the condition being treated; various factors specific to the medical history of the subject to whom the dosage is administered such as the age; weight, sex, extent of disorder and general physical condition of the particular subject, as well as other medication the individual may be taking; as is well known to those skilled in the art. Furthermore, it is evident that said effective daily amount may be lowered or increased depending on the response of the treated subject and/or depending on the evaluation of the physician prescribing the compounds of the present disclosure.

As already mentioned, the present disclosure also relates to a pharmaceutical composition comprising a disclosed therapeutic agent, a pharmaceutically acceptable salt, a hydrate thereof, a solvate thereof, a polymorph thereof, and one or more other therapeutic agents in the treatment, prevention, control, amelioration, or reduction of risk of a burn, e.g., sunburn, may have utility as well as to the use of such a composition for the manufacture of a medicament. Accordingly, in a further aspect, the present disclosure is further directed to a method for the manufacture of a medicament for treating a burn, e.g., a sunburn, comprising combining one or more disclosed compounds, products, or compositions with a pharmaceutically acceptable carrier or diluent. Thus, in one aspect, the present disclosure further relates to a method for manufacturing a medicament comprising combining at least one disclosed compound or at least one disclosed product with a pharmaceutically acceptable carrier or diluent.

### Methods of Using the Disclosed Pharmaceutical Compositions

In various aspects, the present disclosure pertains to methods of using the disclosed pharmaceutical compositions, the method comprising the step of administering the disclosed pharmaceutical composition to a subject in need thereof, e.g., a patient having a burn, such as a sunburn.

The subject in need thereof can be a patient diagnosed with a cutaneous burn, which can be defined as a thermal attack on the skin. A cutaneous burn can lead to partial or total destruction of the skin, of the soft tissues, of the ears and eyes, of the head hair and body hair, of the nails and even of the bones. Most burns affect only the skin, namely: the epidermis and the dermis. Several factors are taken into account for evaluating the seriousness of a burn, namely: the surface area affected, the depth, the localization and also the cause of the burn. As understood herein, a cutaneous burn is inclusive of a sunburn.

Cutaneous burns are usually listed according to their degrees of seriousness. A first-degree burn is generally limited to an erythema, and affects only the surface layers of the epidermis. A second-degree burn “A” affects the epidermis and also a part of the dermis. These burns manifest themselves through the appearance of erythema and also of phlyctenae at the surface thereof. This type of burn may be responsible for more or less acute pain depending on the degree to which the nerves are affected. The rupturing of blood capillaries may already be associated therewith. A deep second-degree burn “B” can extend beyond the dermis and reach the subcutaneous soft tissues. A third-degree burn causes total destruction or vitrification of the epidermis and of the dermis. This type of burn often damages the subcutaneous tissues such as the vascular tissue, the muscles and the nerves. In order to treat this type of burn, it is customary practice to perform skin grafts since no epidermal cicatrization is theoretically possible. The fourth-degree burn affects the muscles and can even extend to the bones. In this case, the appearance of the skin is said to be “carbonized” and the recommended treatment may be amputation. In this case, the patient's vital prognosis is then often involved depending on the age of the patient and the surface area affected.

Burns can be caused by various causes, such as: contact with a solid, liquid or gaseous heat source (thermal burn), radiation from a heat source (thermal burn), certain medical treatments such as radiotherapy, contact with cold (frostbite), an electrocution (electrical burn), contact with a chemical product (chemical burn), friction (friction burn which is generally likened to a thermal burn), or as a result of dermal procedure such as waxing. In various aspects, the disclosed method comprises administering a disclosed pharmaceutical composition for any disclosed burn type, including, but not limited to, radiation dermatitis, thermal burns (including sunburn), and dermatomyofibromas.

In a further aspect, the disclosed method comprises administering a therapeutically effective amount of a disclosed pharmaceutic composition for the protection of the skin against the effects of UV irradiation, including but not limited to sunburn, skin redness, swelling, immune suppression, photo-aging, photodamage, and skin cancer.

Sunburn is produced by the ultraviolet (UV) radiation of the sun and is mediated by inflammatory substance cyclooxygenase-2 and prostaglandins. Cyclooxygenase-2 expression is induced by ultraviolet irradiation; data suggest that tyrosine kinases and reactive oxygen intermediates are involved in this induction. Isoherranen, K, et al, 140(6) BR J DERMATOL 1017-22 (1999). The effects of ultra-violet radiation of the sun can be mitigated by oral supplementation with dietary fish oils. Following 3 months of fish oil, prostaglandin E2 decreased in both control and irradiated skin. Reduction of UV-induced trauma by fish oil may be due, at least partially, to lowered prostaglandin E2 levels. The photoprotection against UVA provocation of a papular response suggests a clinical application for fish oil in polymorphic light eruption. Rhodes, L E, et al 105(4) J INVEST DERMATOL 532-5 (1995).

Radiation therapy has traditionally been the treatment of choice for locally or regionally advanced cancer, but its therapeutic efficacy is often hindered by limited tolerance of normal tissues and by tumor radio resistance. To improve therapeutic outcome, radiotherapy is frequently combined with chemotherapeutic drugs that are themselves cytotoxic and may sensitize cells to radiation. Milas L, et al, 17 (5 Suppl 5) ONCOLOGY (Hunting) 15-24 (2003). Radiation may cause severe burns of the skin and surrounding tissue as well as permanent changes in pigmentation. As many as 95% of patients treated with radiation therapy for cancer will experience a skin reaction. Porock D, Nikoletti S, Kristjanson L 19(4) PLAST SURG NURS.185-92 (1999).

Some patients suffer radiation induced skin injuries and younger patients may face an increased risk of future cancer. Davis M M, et al 20(4) J AM ACAD DERMATOL 608-16 (1989). Interventionists are suffering injury and are exposing their staff to high doses of radiation. In some interventional procedures, skin doses to staff approach those experienced in some cancer radiotherapy fractions. Radiation induced skin injuries occur in patients due to the use of inappropriate equipment and, more often, poor operational technique. Valentin, 30(2) J. ANN ICRP, 7-67 (2000). Others report that such burns, when experienced in the hand, require aggressive debridement and immediate coverage with well vascularized flaps, either regional or free-tissue transfers to achieve adequate wound healing and the most rapid, effective return of function with rapid institution of therapeutic modalities. Milanov N O, Shilov B L, Tjulenev A V 92(2) PLAST RECONSTR SURG. 294-300(1993).

In a further aspect, treatment of radiation dermatitis according to the disclosure is performed by topical application of a disclosed pharmaceutical compositions to the affected area immediately after radiation treatments and is repeated from once to three times daily throughout the course of radiation, specifically twice daily.

In a further aspect, treatment of superficial burn according to the disclosure is performed by topical application of the compositions of the disclosure to the affected area immediately after the burn is experienced and repeated from one to three times daily until the skin heals.

In a further aspect, treatment of dermatomyofibromas according to the disclosure is performed by topical application of the compositions to the lesions as soon as a new lesion is noted and repeated from one to five times daily until the skin heals, specifically three times daily, until the skin heals.

In a further aspect, prevention and treatment of exposure-induced wrinkles according to the disclosure is performed by topical application of the compositions of the disclosure to the skin prior to exposure, e.g., wind, sun, and after exposure one to three times daily as needed. In view of the above description and the examples below, one of ordinary skill in the art will be able to practice the disclosure as claimed without undue experimentation. The foregoing will be better understood with reference to the following examples that detail certain procedures for the preparation of formulations according to the present disclosure. All references made to these examples are for the purposes of illustration. The following examples should not be considered exhaustive, but merely illustrative of only a few of the many aspects contemplated by the present disclosure.

In various aspects, the present disclosure pertains to methods of using the disclosed pharmaceutical compositions, the method comprising the step of administering the disclosed pharmaceutical composition to a subject in need thereof, e.g., a patient having pain in one or more joints, such as knee, ankle, hip, elbow, shoulder, wrist, neck, one or more fingers, one or more toes, or a combination thereof.

Joint pain can be acute musculoskeletal pain such as a sprain or muscle pull. In one aspect, acute musculoskeletal pain can improve over the course of one week, two weeks, or three weeks, without treatment, but acute musculoskeletal pain can greatly impact quality of life for its duration. In another aspect, joint pain can be chronic pain such as pain related to an arthritic condition, long-term cancer treatment, or the like. In one aspect, long-term use of oral non-steroidal anti-inflammatory drugs may be associated with a greater incidence of upper gastrointestinal bleeding. Further in this aspect, use of topical compositions as disclosed herein can reduce the incidence of such bleeding.

The subject in need thereof can be a patient diagnosed with an injury such as a repetitive strain or overuse injury, a broken bone, a sprain, a dislocation, or tendinitis, with one or more subtypes of arthritis such as gonococcal arthritis, gout or pseudogout, juvenile idiopathic arthritis, osteoarthritis, psoriatic arthritis, reactive arthritis, rheumatoid arthritis, or septic arthritis, with another bone condition or disease such as avascular necrosis, bone cancer, Paget's disease, or osteomyelitis, with another cancer such as leukemia, with a nutritional deficiency such as rickets, with a glandular disorder such as hypothyroidism, with an autoimmune or inflammatory condition such as ankylosing spondylitis, bursitis, lupus, fibromyalgia, polymyalgia rheumatica, or sarcoidosis, with another syndrome such as complex regional pain syndrome, or a combination of any of the above. Joint pain can manifest when various parts of the joint are infected, injured, or otherwise inflamed or may be an effect of a nervous system or glandular dysfunction. As understood herein, joint pain can be temporary, as with an injury or nutritional deficiency, or can be a long-term condition such as joint pain associated with arthritis or an autoimmune disease or disorder. Furthermore, joint pain can affect joints of any size and can interfere with subject's quality of life, motor functions, mobility, and the like.

Fungal infection as used herein can refer to a fungal infection on any surface of the body accessible for application of a topical preparation including, but not limited to, the foot and/or toenails, fingernails, body skin, scalp skin, groin, in or near a wound, or any combination thereof. In one aspect, fungal infection can cause an itching or burning sensation in a subject, or can cause welts or pustules, can cause hair loss, discoloration and/or thickening of the nails, or any combination thereof. In one aspect, effects of a fungal infection can be cosmetic. In another aspect, effects of a fungal infection can cause discomfort. In some aspects, fungal infections in one area (e.g., the feet) can spread to nearby areas (e.g., toenails), especially in the event of an injury. In some aspects, treatment of a cutaneous bacterial infection can leave the skin susceptible to opportunistic or follow-on fungal infections caused by fungal species that typically colonize the skin without causing infection. In other aspects, when a subject scratches at a fungal infection, this can cause the fungal infection to spread to areas subsequently touched, or can cause abrasions in the skin that may then be susceptible to bacterial infection. In one aspect, fungal infections can be especially problematic in immunocompromised subjects and/or those with circulatory disorders, diabetes, and the like. In another aspect, oral antifungal agents can cause severe systemic side effects and may be contra-indicated in some subjects, whereas topical antifungal agents can still be used in individuals in whom oral antifungal agents are not advisable.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### Aspects

Aspect 1: A pharmaceutical composition comprising a dermal restorative agent, a pain modulator agent, a soothing agent, an anti-inflammatory agent, and a dermal health agent, or a pharmaceutically acceptable salt thereof each therapeutic agent, and pharmaceutically acceptable excipient or carrier.

Aspect 2: The pharmaceutical composition of Aspect 1, wherein the dermal restorative agent comprises hyaluronic acid or the pharmaceutically-acceptable salt thereof.

Aspect 3: The pharmaceutical composition of Aspect 2, wherein the hyaluronic acid has weight-average molecular weight of between about 0.1 and about 1000 kDa.

Aspect 4: The pharmaceutical composition in any one of Aspects 1-3, wherein the dermal restorative agent is from about 0.1 to about 10 wt % of the pharmaceutical composition.

Aspect 5: The pharmaceutical composition in any one of Aspects 1-4, wherein the pain modulator agent comprises an anesthetic agent.

Aspect 6: The pharmaceutical composition in any one of Aspects 1-4, wherein the pain modulator agent comprises butacaine, chloroprocaine, cocaine, cyclomethycaine, hexylcaine, procaine, proparacaine, propoxycaine, tetracaine, benzocaine, bupivacaine, dibucaine, etidocaine, lidocaine, mepivacaine, ropivacaine, prilocaine, dyclonine, pramoxine, lidocaine, prilocaine, scandicaine, etidocaine, bupivacaine, ropivacaine, levobupivacaine, articaine, mepivacaine, and the pharmaceutically acceptable salts thereof, and any combination thereof.

Aspect 7: The pharmaceutical composition in any one of Aspects 1-6, wherein the pain modulator agent is from about 1 wt % to about 20 wt % of the pharmaceutical composition.

Aspect 8: The pharmaceutical composition in any one of Aspects 1-7, wherein the soothing agent comprises an aloe gel, aloe paste, aloe extract, aloe powder, or any combination thereof.

Aspect 9: The pharmaceutical composition in any one of Aspects 1-7, wherein the soothing agent is from about 1 wt % to about 75 wt % of the pharmaceutical composition.

Aspect 10: The pharmaceutical composition in any one of Aspects 1-9, wherein the anti-inflammatory agent comprises a non-steroidal anti-inflammatory drug (NSAID).

Aspect 11: The pharmaceutical composition in any one of Aspects 1-9, wherein the anti-inflammatory agent comprises a COX-2 specific inhibitor.

Aspect 12: The pharmaceutical composition in any one of Aspects 1-11, wherein the anti-inflammatory agent is from about 0.1 wt % to about 10 wt % of the pharmaceutical composition.

Aspect 13: The pharmaceutical composition in any one of Aspects 1-12, wherein dermal health agent comprises an antioxidant.

Aspect 14: The pharmaceutical composition in any one of Aspects 1-12, wherein dermal health agent comprises Vitamin A, Vitamin E, Vitamin C, Vitamin K, Vitamin B3, green tea, resveratrol, curcumin, lycopene, coenzyme q10, a polyphenol, isebenone, grape seed, or any combination thereof.

Aspect 15: The pharmaceutical composition in any one of Aspects 1-14, further comprising a disclosed secondary agent comprising calendula oil, cucumber oil, witch hazel oil or extract, lavender oil, olive oil, coconut oil, capryl caprylate triglyceride, shea butter, D-panthenol, glycerine, allantoin, oatmeal extract, or combination thereof.

Aspect 16: The pharmaceutical composition in any one of Aspects 1-15, further comprising an antifungal agent.

Aspect 17: The pharmaceutical composition of Aspect 16, wherein the antifungal agent comprises bifonazole, clotrimazole, econazole, efinaconazole, ketoconazole, luliconazole, miconazole, sulconazole, tioconazole, terbinafine, tolciclate, tolnaftate, tavaborole, amorolfine, butenafine, oxiconazole, nystatin, voriconazole, itraconazole, caspofungin, micafungin, naftifine, butenafine, amorolfine, ravuconazole, posaconazole, flucytosine, sertaconazole, efinaconazole, enilaconazole, saperconazole, terconazole, nikkomycin Z, anidulafungin (LY303366), pimaricin, griseofulvin, ciclopirox, haloprogin, tolnaftate, undecylenate,or any combination thereof.

Aspect 18: The pharmaceutical composition in any one of Aspects 1-17, wherein the antifungal is from about 0.1 wt % to about 10 wt % of the pharmaceutical composition.

Aspect 19: The pharmaceutical composition in any one of Aspects 1-18, further comprising an anti-microbial agent.

Aspect 20: The pharmaceutical composition of Aspect 19, wherein the anti-microbial agent comprises an antiseptic, an antibiotic, or a combination thereof.

Aspect 21: The pharmaceutical composition in any one of Aspects 1-20, further comprising silver or a silver salt.

Aspect 22: The pharmaceutical composition of Aspect 21, wherein the silver or silver salt comprises silver nitrate, silver sulfadiazine, metallic silver, colloidal silver, silver chloride, silver salicylic acid, silver-calcium-sodium phosphate, silver charcoal, silver zeolite, nanocrystalline silver, or any combination thereof.

Aspect 23: The pharmaceutical composition of Aspect 21, wherein the silver or silver salt is from about 0.1 wt % to about 10 wt % of the pharmaceutical composition.

Aspect 24: The pharmaceutical composition of Aspect 1, wherein the composition comprises (i) an antifungal agent and (ii) silver or a silver salt.

Aspect 25: A pharmaceutical composition comprising (i) an antifungal agent and (ii) silver or a silver salt.

Aspect 26: The pharmaceutical composition of Aspect 25, wherein the antifungal agent comprises bifonazole, clotrimazole, econazole, efinaconazole, ketoconazole, luliconazole, miconazole, sulconazole, tioconazole, terbinafine, tolciclate, tolnaftate, tavaborole, amorolfine, butenafine, oxiconazole, nystatin, voriconazole, itraconazole, caspofungin, micafungin, naftifine, butenafine, amorolfine, ravuconazole, posaconazole, flucytosine, sertaconazole, efinaconazole, enilaconazole, saperconazole, terconazole, nikkomycin Z, anidulafungin (LY303366), pimaricin, griseofulvin, ciclopirox, haloprogin, tolnaftate, undecylenate,or any combination thereof.

Aspect 27: The pharmaceutical composition of Aspect 25 or 26, wherein the antifungal is from about 0.1 wt % to about 10 wt % of the pharmaceutical composition.

Aspect 28: The pharmaceutical composition in any one of Aspects 25-27, wherein the silver or silver salt comprises silver nitrate, silver sulfadiazine, metallic silver, colloidal silver, silver chloride, silver salicylic acid, silver-calcium-sodium phosphate, silver charcoal, silver zeolite, nanocrystalline silver, or any combination thereof.

Aspect 29: The pharmaceutical composition in any one of Aspects 25-28, wherein the silver or silver salt is from about 0.1 wt % to about 10 wt % of the pharmaceutical composition.

Aspect 30: The pharmaceutical composition in any one of Aspects 25-29, wherein the antifungal agent comprises tolnaftate and the silver salt comprises silver nitrate.

Aspect 31: The pharmaceutical composition in any one of Aspects 25-29, wherein the antifungal agent comprises butenafine and the silver salt comprises silver nitrate.

Aspect 32: A method of treating a burn in a subject comprising the step of administering the pharmaceutical composition of any of Aspects 1-31.

Aspect 33: The method of Aspect 32, wherein the burn is a sunburn.

Aspect 34: A method of treating joint pain in a subject comprising the step of administering the pharmaceutical composition of any of Aspects 1-31.

Aspect 35: The method of Aspect 34, wherein the joint pain occurs in the knee, ankle, hip, elbow, shoulder, wrist, neck, one or more fingers, one or more toes, or a combination thereof.

Aspect 36: A method of treating a fungal infection in a subject comprising the step of administering the pharmaceutical composition of any of Aspects 1-31.

Aspect 37: The method of Aspect 36, wherein the fungal infection occurs on the skin, feet, nails, scalp, groin, or in a wound, or a combination thereof.

Aspect 38: A kit comprising: a dermal restorative agent, a pain modulator agent, a soothing agent, an anti-inflammatory agent, and a dermal health agent, or a pharmaceutically acceptable salt thereof each therapeutic agent, a pharmaceutically acceptable excipient or carrier, and instructions for preparation of a topical pharmaceutical composition comprising the foregoing or instructions for the use of the foregoing for treatment of a burn, or joint pain.

Aspect 39: The kit of Aspect 38, further comprising silver or a silver salt.

Aspect 40: A kit comprising: a dermal restorative agent, a pain modulator agent, a soothing agent, an anti-inflammatory agent, and a dermal health agent, or a pharmaceutically acceptable salt thereof each therapeutic agent, a pharmaceutically acceptable excipient or carrier, and instructions for preparation of a topical pharmaceutical composition comprising the foregoing or instructions for the use of the foregoing for treatment of a fungal infection.

Aspect 41: The kit of Aspect 40, further comprising an antifungal agent.

Aspect 42: The kit of Aspect 40 or 41, further comprising silver or a silver salt.

Aspect 43: A kit comprising: an antifungal agent or a pharmaceutically acceptable salt thereof, silver or a silver salt, a pharmaceutically acceptable excipient or carrier, and instructions for preparation of a topical pharmaceutical composition comprising the foregoing or instructions for the use of the foregoing for treatment of a fungal infection.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Prospective Example 1

A study comprising 15-24 subjects for a period of up to four weeks will be conducted to test the hypothesis that a disclosed pharmaceutical compositions comprising disclosed therapeutic agents when mixed and combined in appropriate proportions within a topical cream and applied to the surface of the moderately burned skin will accelerate the reduction of discomfort and enhance the epithelization of the skin. The study will assess end points such as patient comfort, patient satisfaction, pain reduction, redness reduction, and re-epithelialization. The endpoints will be assessed as follows: (a) time taken to improved re-epithelialization; (b) pain during recovery process, utilizing a pain rating scale; (c) number of dressing changes; (d) patient satisfaction utilizing a patient satisfaction rating scale; and (e) incidence of adverse events. It is anticipated that the clinical study will determine an improvement in one or more of the foregoing endpoints compared to a conventional product indicated for the same or similar therapeutic indication. Moreover, it is believed that the study can provide data regarding the synergistic effect of the therapeutic agents and secondary agents.

### Prospective Example 2

Formulations for treating pain will be prepared as shown in Tables 2 and 3:

TABLE 2
Pain Relief Formulation

| Ingredient No. | Ingredient Identity | Amount (wt. %) |
|---|---|---|
| 1 | Sodium Hyaluronate | 1.00 |
| 2 | Pramoxine | 0.80 |
| 3 | Allantoin | 0.30 |
| 4 | Vitamin E | 0.08 |
| 5 | Aloe Vera Liquid Extract | 3.00 |
| 6 | Calendula Oil | 0.50 |
| 7 | Cucumber Oil | 2.00 |
| 8 | Witch Hazel Extract | 2.00 |
| 9 | Lavender Oil | 0.50 |
| 10 | Olive Oil | 0.50 |
| 11 | Capryl Caprylate Triglyceride | 1.50 |
| 12 | D-Panthenol | 0.50 |
| 13 | Shea Butter | 2.00 |
| 14 | Glycerine | 3.00 |
| 15 | Oatmeal Extract | 0.50 |
| 16 | LEXGARD ® Natural MB | 1.00 |
| 17 | SEPINOV ™ EMT 10 | 1.00 |
| 18 | Vitamin K | Variable |
| 19 | Purified Water | To 100% |

The base formulation can be modified with additional active and inactive ingredients for treatment of other conditions.

TABLE 3
Pain Relief Formulation

| Ingredient No. | Ingredient Identity | Amount (wt. %) |
|---|---|---|
| 1 | Sodium Hyaluronate | 0.5 |
| 2 | Pramoxine HCl | 0.5 |
| 3 | Allantoin | 4 |
| 4 | Polysorbate 80 | 1.5 |
| 5 | Glycerine | 3 |
| 6 | Aloe Vera | 1.3 |
| 7 | Witch Hazel Extract | 0.5 |
| 8 | Shea Butter | 1.5 |
| 9 | Vitamin E | 0.1 |
| 10 | Capryl Caprylate Triglyceride | 1.5 |
| 11 | Oatmeal Extract | 0.5 |
| 12 | Cucumber Oil | 2 |
| 13 | D-Panthenol | 0.5 |
| 14 | Lavender Oil | 0.5 |
| 15 | Cetostearyl Alcohol | 6 |
| 16 | Glycerol Monostearate | 3 |
| 17 | Cetyl Alcohol | 2.5 |
| 18 | SPECTRASTAT ™ Natural MB | 1 |
| 19 | SEPINOV ™ EMT 10 | 0.5 |
| 20 | Fragrance | 0.5 |
| 21 | Purified Water | 68.6 |

### Prospective Example 3

Formulations for treating athlete's foot will be prepared as shown in Tables 4 and 5, based on modifications of the example compositions in Tables 2 and 3:

TABLE 4
Athlete's Foot Formulation

| Ingredient No. | Ingredient Identity | Amount (wt. %) |
|---|---|---|
| 1 | Sodium Hyaluronate | 2.00 |
| 2 | Pramoxine | 0.90 |
| 3 | Allantoin | 0.40 |
| 4 | Vitamin E | 0.09 |
| 5 | Aloe Vera Liquid Extract | 4.00 |
| 6 | Tolnaftate | Variable |
| 7 | Calendula Oil | 0.50 |
| 8 | Cucumber Oil | 2.00 |
| 9 | Witch Hazel Extract | 2.00 |
| 10 | Lavender Oil | 0.50 |
| 11 | Olive Oil | 0.50 |
| 12 | Capryl Caprylate Triglyceride | 2.00 |
| 13 | D-Panthenol | 0.50 |
| 14 | Shea Butter | 2.00 |
| 15 | Glycerine | 3.00 |
| 16 | Oatmeal Extract | 0.50 |
| 17 | LEXGARD ® Natural MB | 1.00 |
| 18 | SEPINOV ™ EMT 10 | 0.70 |
| 19 | Vitamin K | Variable |
| 20 | Purified Water | To 100% |

TABLE 5
Athlete's Foot Formulation

| Ingredient No. | Ingredient Identity | Amount (wt. %) |
|---|---|---|
| 1 | Sodium Hyaluronate | 1 |
| 2 | Pramoxine HCl | 1 |
| 3 | Allantoin | 0.8 |
| 4 | Polysorbate 80 | 1.5 |
| 5 | Glycerine | 3 |
| 6 | Aloe Vera | 3 |
| 7 | Witch Hazel Extract | 0.5 |
| 8 | Shea Butter | 1.5 |
| 9 | Vitamin E | 0.1 |
| 10 | Capryl Caprylate Triglyceride | 1.5 |
| 11 | Oatmeal Extract | 0.5 |
| 12 | Cucumber Oil | 2 |
| 13 | D-Panthenol | 0.5 |
| 14 | Lavender Oil | 0.5 |
| 15 | Cetostearyl Alcohol | 6 |
| 16 | Glycerol Monostearate | 3 |
| 17 | Cetyl Alcohol | 2.5 |
| 18 | SPECTRASTAT ™ Natural MB | 1 |
| 19 | SEPINOV ™ EMT 10 | 0.5 |
| 20 | Fragrance | 0.5 |
| 21 | Tolnaftate | 1 |
| 22 | Silver | 1 |
| 23 | Purified Water | 67.1 |

### Prospective Example 4

Formulations for treating moderate burns will be prepared as shown in Tables 6 and 7, based on modifications of the example compositions in Tables 2 and 3:

TABLE 6
Burn Formulation

| Ingredient No. | Ingredient Identity | Amount (wt. %) |
|---|---|---|
| 1 | Sodium Hyaluronate | 3.00 |
| 2 | Pramoxine | 1.00 |
| 3 | Allantoin | 0.50 |
| 4 | Vitamin E | 0.10 |
| 5 | Aloe Vera Liquid Extract | 5.00 |
| 6 | Silver | Variable |
| 7 | Calendula Oil | 0.50 |
| 8 | Cucumber Oil | 2.00 |
| 9 | Witch Hazel Extract | 2.00 |
| 10 | Lavender Oil | 0.50 |
| 11 | Olive Oil | 0.50 |
| 12 | Capryl Caprylate Triglyceride | 2.50 |
| 13 | D-Panthenol | 0.50 |
| 14 | Shea Butter | 2.00 |
| 15 | Glycerine | 3.00 |
| 16 | Oatmeal Extract | 0.50 |
| 17 | LEXGARD ® Natural MB | 1.00 |
| 18 | SEPINOV ™ EMT 10 | 0.50 |
| 19 | Vitamin K | Variable |
| 20 | Purified Water | To 100% |

TABLE 7
Burn Formulation

| Ingredient No. | Ingredient Identity | Amount (wt. %) |
|---|---|---|
| 1 | Sodium Hyaluronate | 1.25 |
| 2 | Pramoxine HCl | 1 |
| 3 | Allantoin | 0.3 |
| 4 | Polysorbate 80 | 1.5 |
| 5 | Glycerine | 3 |
| 6 | Aloe Vera | 3 |
| 7 | Witch Hazel Extract | 0.5 |
| 8 | Shea Butter | 1.5 |
| 9 | Vitamin E | 0.1 |
| 10 | Capryl Caprylate Triglyceride | 1.5 |
| 11 | Oatmeal Extract | 0.5 |
| 12 | Cucumber Oil | 2 |
| 13 | D-Panthenol | 0.5 |
| 14 | Lavender Oil | 0.5 |
| 15 | Cetostearyl Alcohol | 6 |
| 16 | Glycerol Monostearate | 3 |
| 17 | Cetyl Alcohol | 2.5 |
| 18 | SPECTRASTAT ™ Natural MB | 1 |
| 19 | SEPINOV ™ EMT 10 | 0.5 |
| 20 | Fragrance | 0.5 |
| 21 | Purified Water | 69.35 |

It should be emphasized that the above-described aspects of the present disclosure are merely possible examples of implementations set forth for a clear understanding of the principles of the disclosure. Many variations and modifications may be made to the above-described aspect(s) without departing substantially from the spirit and principles of the disclosure. All such modifications and variations are intended to be included herein within the scope of this disclosure and protected by the following claims.

What is claimed is:

1. A pharmaceutical composition comprising a dermal restorative agent, a pain modulator agent, a soothing agent, an anti-inflammatory agent, and a dermal health agent, or a pharmaceutically acceptable salt thereof each therapeutic agent, and pharmaceutically acceptable excipient or carrier.

2. The pharmaceutical composition of claim 1, wherein the dermal restorative agent comprises hyaluronic acid or the pharmaceutically-acceptable salt thereof.

3. The pharmaceutical composition of claim 2, wherein the hyaluronic acid has weight-average molecular weight of between about 0.1 and about 1000 kDa.

4. The pharmaceutical composition of claim 1, wherein the dermal restorative agent is from about 0.1 to about 10 wt % of the pharmaceutical composition.

5. The pharmaceutical composition of claim 1, wherein the pain modulator agent comprises an anesthetic agent.

6. The pharmaceutical composition of claim 1, wherein the pain modulator agent comprises butacaine, chloroprocaine, cocaine, cyclomethycaine, hexylcaine, procaine, proparacaine, propoxycaine, tetracaine, benzocaine, bupivacaine, dibucaine, etidocaine, lidocaine, mepivacaine, ropivacaine, prilocaine, dyclonine, pramoxine, lidocaine, prilocaine, scandicaine, etidocaine, bupivacaine, ropivacaine, levobupivacaine, articaine, mepivacaine, and the pharmaceutically acceptable salts thereof, and any combination thereof.

7. The pharmaceutical composition of claim 1, wherein the pain modulator agent is from about 1 wt % to about 20 wt % of the pharmaceutical composition.

8. The pharmaceutical composition of claim 1, wherein the soothing agent comprises an aloe gel, aloe paste, aloe extract, aloe powder, or any combination thereof.

9. The pharmaceutical composition of claim 1, wherein the soothing agent is from about 1 wt % to about 75 wt % of the pharmaceutical composition.

10. The pharmaceutical composition of claim 1, wherein the anti-inflammatory agent comprises a non-steroidal anti-inflammatory drug (NSAID) or a COX-2 specific inhibitor.

11. The pharmaceutical composition of claim 1, wherein the anti-inflammatory agent is from about 0.1 wt % to about 10 wt % of the pharmaceutical composition.

12. The pharmaceutical composition of claim 1, wherein dermal health agent comprises an antioxidant.

13. The pharmaceutical composition of claim 1, wherein dermal health agent comprises Vitamin A, Vitamin E, Vitamin C, Vitamin K, Vitamin B3, green tea, resveratrol, curcumin, lycopene, coenzyme q10, a polyphenol, isebenone, grape seed, or any combination thereof.

14. The pharmaceutical composition of claim 1, further comprising a disclosed secondary agent comprising calendula oil, cucumber oil, witch hazel oil or extract, lavender oil, olive oil, coconut oil, capryl caprylate triglyceride, shea butter, D-panthenol, glycerine, allantoin, oatmeal extract, or combination thereof.

15. The pharmaceutical composition of claim 1, further comprising an antifungal agent.

16. The pharmaceutical composition of claim 15, wherein the antifungal agent comprises bifonazole, clotrimazole, econazole, efinaconazole, ketoconazole, luliconazole, miconazole, sulconazole, tioconazole, terbinafine, tolciclate, tolnaftate, tavaborole, amorolfine, butenafine, oxiconazole, nystatin, voriconazole, itraconazole, caspofungin, micafungin, naftifine, butenafine, amorolfine, ravuconazole, posaconazole, flucytosine, sertaconazole, efinaconazole, enilaconazole, saperconazole, terconazole, nikkomycin Z, anidulafungin (LY303366), pimaricin, griseofulvin, ciclopirox, haloprogin, tolnaftate, undecylenate,or any combination thereof.

17. The pharmaceutical composition of claim 15, wherein the antifungal is from about 0.1 wt % to about 10 wt % of the pharmaceutical composition.

18. The pharmaceutical composition of claim 1, further comprising an anti-microbial agent, wherein the anti-microbial agent comprises an antiseptic, an antibiotic, or a combination thereof.

19. The pharmaceutical composition of claim 1, further comprising silver or a silver salt.

20. The pharmaceutical composition of claim 19, wherein the silver or silver salt comprises silver nitrate, silver sulfadiazine, metallic silver, colloidal silver, silver chloride, silver salicylic acid, silver-calcium-sodium phosphate, silver charcoal, silver zeolite, nanocrystalline silver, or any combination thereof.

21. The pharmaceutical composition of claim 19, wherein the silver or silver salt is from about 0.1 wt % to about 10 wt % of the pharmaceutical composition.

22. The pharmaceutical composition of claim 1, wherein the composition comprises (i) an antifungal agent and (ii) silver or a silver salt.

23. A pharmaceutical composition comprising (i) an antifungal agent and (ii) silver or a silver salt.

24. The pharmaceutical composition of claim 23, wherein the antifungal agent comprises bifonazole, clotrimazole, econazole, efinaconazole, ketoconazole, luliconazole, miconazole, sulconazole, tioconazole, terbinafine, tolciclate, tolnaftate, tavaborole, amorolfine, butenafine, oxiconazole, nystatin, voriconazole, itraconazole, caspofungin, micafungin, naftifine, butenafine, amorolfine, ravuconazole, posaconazole, flucytosine, sertaconazole, efinaconazole, enilaconazole, saperconazole, terconazole, nikkomycin Z, anidulafungin (LY303366), pimaricin, griseofulvin, ciclopirox, haloprogin, tolnaftate, undecylenate,or any combination thereof.

25. The pharmaceutical composition of claim 23, wherein the antifungal is from about 0.1 wt % to about 10 wt % of the pharmaceutical composition.

26. The pharmaceutical composition of claim 23, wherein the silver or silver salt comprises silver nitrate, silver sulfadiazine, metallic silver, colloidal silver, silver chloride, silver salicylic acid, silver-calcium-sodium phosphate, silver charcoal, silver zeolite, nanocrystalline silver, or any combination thereof.

27. The pharmaceutical composition of claim 23, wherein the silver or silver salt is from about 0.1 wt % to about 10 wt % of the pharmaceutical composition.

28. The pharmaceutical composition of claim 23, wherein the antifungal agent comprises butenafine or tolnaftate and the silver salt comprises silver nitrate.

29. A method of treating a burn or joint pain in a subject comprising the step of administering the pharmaceutical composition of claim 1.

30. The method of claim 29, wherein the burn is a sunburn.

31. The method of claim 29, wherein the joint pain occurs in the knee, ankle, hip, elbow, shoulder, wrist, neck, one or more fingers, one or more toes, or a combination thereof.

32. A method of treating a fungal infection in a subject comprising the step of administering the pharmaceutical composition of claim 1.

33. The method of claim 32, wherein the fungal infection occurs on the skin, feet, nails, scalp, groin, or in a wound, or a combination thereof.
